(19) United States
(12) Patent Application Publication
(10) Pub. No.: US 2016/0256280 A1
(43) Pub. Date: Sep. 8, 2016

(54) BONE IMPLANT AUGMENT METHOD AND APPARATUS

(71) Applicant: Kenneth B. Trauner, San Francisco, CA (US)

(72) Inventor: Kenneth B. Trauner, San Francisco, CA (US)

(21) Appl. No.: 15/059,511

(22) Filed: Mar. 3, 2016

Related U.S. Application Data
(60) Provisional application No. 62/128,732, filed on Mar. 5, 2015.
(60) Provisional application No. 62/133,072, filed on Mar. 13, 2015.
(60) Provisional application No. 62/237,018, filed on Oct. 5, 2015.

(51) Int. Cl. A61F 2/30; A61F 2/46

(52) U.S. Cl. CPC A61F 2/30734; A61F 2/4601; A61F 2002/30065; A61F 2002/30583; A61F 2002/30736

(57) ABSTRACT

An offset of a bone implant relative to a bone can be adjusted using cured PMMA inserts. One or more PMMA inserts can be used to change the offset distance and/or angle of a implant relative to the bone. During a bone implant surgery, if excessive bone has been removed, PMMA inserts can be inserted into the bone to build up the resection surface of the bone. A trial implant can be placed on the PMMA inserts to trial the offset. When the trial is passed, liquid PMMA cement can be applied to form a chemical bond with the PMMA inserts and forma mechanical bond between the implant and the bone.

### CROSS REFERENCE TO RELATED APPLICATIONS

This patent application claims priority to: U.S. Provisional Patent Application No. 62/128,732, “PMMA Shims For Total Knee Arthroplasty” filed Mar. 5, 2015, U.S. Provisional Patent Application No. 62/133,072, “PMMA Shims For Total Knee Arthroplasty” filed Mar. 13, 2015, and U.S. Provisional Patent Application No. 62/237,018, “Shim Augment System” filed Oct. 5, 2015, both of which are incorporated by reference in their entireties.

### BACKGROUND

The proper functioning of a joint, such as the knee, hip, shoulder, ankle or elbow can be impeded by a variety of factors, including, disease, such as osteoarthritis, mechanical injury, bone deformation and a variety of other factors. Arthroplasty, or the surgical restoration of a joint, is a known procedure that is often used to relieve pain and improve joint function by replacing the diseased or damaged articulating surfaces of a joint with prosthetic components. Achieving stable joint balance is a primary goal for arthroplasty surgeons. A balanced joint is a joint that has the proper articulation and ligamentous balance in all orientations of the joint. The patient may be most comfortable when the artificial joint replicates the kinematics of the original, natural joint.

One of the most common arthroplasty procedures is knee replacement surgery. Some common forms of knee replacement surgery include total knee replacement (“TKR”) surgery; partial knee replacement surgery, which is also known as unicompartmental arthroplasty (“UKA”); and revision knee surgery. Generally, in a TKR, the femur's bone from the lateral and medial condyles, or the articulating surfaces at the femur's distal end, are removed and replaced with a femoral prosthetic component. Additionally, in a TKR, the tibial plateau at the tibia's proximal end is also removed and replaced with a tibial prosthetic component.

The stability of a total knee arthroplasty is based upon the correct amount of bone resection from the femur and tibia of the knee and the balancing or release of soft tissues about the joint. Determining the correct amount of bone to resect can be challenging in the presence of preoperative bone loss or deformity. The surgeon frequently is forced to guess as to the correct amount of bone to resect. The surgeon performs releases of tissues to improve balance in the knee, especially if the patient has developed contractures prior to the procedure. The surgeon then performs trial range of motion and stressing of the knee with trial implants to determine knee stability. If the balance is not adequate the surgeon has several choices for improving balance. Depending on the type of imbalance, the surgeon can recut and remove bone from any surface, can perform soft tissue balancing or can add increased thickness to plastic tibial liner.

There are several situations in total knee arthroplasty that are not currently well addressed. When too much bone is removed from the distal femur as occurs with knees where bone collapse has occurred preoperatively, the surgeon has limited technical options for addressing the deficiency. Too much bone resection leads to the clinical problem of hyperextension of the knee or if the angle of the resection is incorrect leads to the clinical problem of instability of the knee. The desired solution is to correct the position of the femoral component by building up the implant from the bone surface. The correction may need to be longitudinal, i.e. moving the femur distally and symmetrically from the boney cuts or may be asymetric, i.e. moving the angle of the femur relative to the boney resection surface, with or without longitudinal displacement of the implant.

Currently there are no reliable and efficient techniques for producing these changes for the femoral component.

There are increasing pressures on surgeons to perform efficiently, i.e., perform a large number of procedures each surgical day. These pressures have forced surgeons to optimize their techniques and work flows to minimize delays. Given the variability of patients conditions, numerous variations in patient anatomy and soft tissues need to be addressed in total joint surgery. The surgeons require a system that allows for the quick correction of bone deformities, the assessment of the correction, and quick adjustment to a new correction if the first correction is incorrect, then an ability to proceed with the case without delay once the correction has occurred.

In total knee arthroplasty, once the surgeon has resected the boney surfaces and has balanced the soft tissues, the surgeon routinely will trial the components. The trial components are placed onto the boney surfaces and the knee is taken through range of motion. Throughout motion, the surgeon stresses the knee to test the ligamentous stability especially to varus and valgus stress. It is critical the knee exhibit full range of motion, i.e. that the knee is able to achieve full extension and at least 120 degrees of flexion while exhibiting stability to stress throughout that motion. At this point in the case, if there is not adequate stability or motion, the surgeon will address additional soft tissue balancing or boney cuts.

This invention describes a system for correcting incorrect boney cuts with an implant that can then be trialed, then exchanged if the implant is incorrect and then left in place for the remainder of the procedure once the deficiency has been corrected at the trial stage.

Total knee arthroplasty in recent years has demanded greater and greater accuracy on the part of the surgeon. Robotic systems have also been introduced at great cost to improve reliability of boney cuts. Implants now come with one and 2 to mm increments in sizing. Such accuracy has led to improved clinical results. However, there remains a need to address bone deficiencies in total knee arthroplasty with similar level of accuracy with a simple flexible system that can work reliably, accurately and with minimal alteration in surgical technique. What is needed is a system that can allow a surgeon to make adjustments to the axial length and/or angular alignment of the implant. Such a system is required that allows the surgeon to quickly and easily apply and test the correction, if incorrected quickly address the correction and when corrected, proceed with remainder of the case.

### SUMMARY OF THE INVENTION

The invention is a system for adjusting the length and angle of bone implants relative to the bone during surgeries. For example, a total knee replacement surgery involves cutting (resectioning) an end of the bone and then bonding a knee implant to the end of the bone and then reassembling the knee. The implant which can be a metal structure that is secured by mechanical bonding to the resection surface of the bone with a liquid (Polymethyl methacrylate) PMMA cement. The liquid PMMA cement is applied to the bone and implant in liquid form which structurally bonds the implant to the bone when the liquid PMMA hardens and cures.

A problem with existing systems is that if there is an error in the resectioning, there can be length and/or angular alignment errors of the knee implant. Without making corrections, the patient will not have a good surgical outcome. Incorrect length of cuts can produce excess joint laxity or increased tightness of the joint both associated with decreased pain and function. Any imbalance of a joint will lead to instability which is associated with pain, inflammation and impaired function. Alternatively, the angle of the knee can be wrong resulting in misalignment of the femur and tibia and/or angular instability of the joint. For example, when the implant is offset too far from the bone, the resulting arthroplasty can be too tight resulting in pain and not enough freedom of rotation. Conversely, if the implant offset is not offset enough the implant can be too loose resulting in joint instability. Laxity of just a few millimeters can result in pain and joint instability.

To solve this problem, the invention uses pre-cured PMMA inserts which are made at least partially of hardened PMMA and have good physical strength. The inserts can include various types of structures that are placed between the implant and the bone. The implant structures can include: tacks, shims, rods and any other suitable insert structures. The cured PMMA inserts can have a stem portion which is inserted into the resectioned surface of the bone and a portion that extends away from the surface of the bone to create an offset for the implant relative to the bone.

In an embodiment, an implant can be secured to a resectioned bone surface. The resectioned bone can be drilled and these stem section of the insert can be inserted into the drilled hole(s). This can be necessary when the insert is being placed into hard bone surfaces. In other embodiments, the inserts can be physically pressed into the surface of the bone without drilling the bone. The pressed insertion of the inserts can be useful when the exposed bony surface of the bone is soft. For example, the soft exposed bony surface of the bone: a metaphyseal bone, a cancellous bone, a trabecular bone, or a porous bone. By manually inserting the PMMA inserts, the surgeon can more easily control the positions and angles of the inserts.

Once the inserts are placed in the bone, the surgeon can then check the position of the implant against or adjacent to the insert(s) to determine the offset of the implant relative to the bone. A trial implant can be placed against the insert(s) and a trial assessment can be performed which can include checking the range of motion and stability of the joint with the trial implant. The trial implants can provide all of the function needed for the trial assessment without having to use the final implant. In other embodiments, rather than using the trial implants, the alignment provided by the inserts can be checked with an alignment template to determine if the insert(s) will provide the proper implant length or angular offset.

The surgeon can check the functional correction of the joint with trial implants placed against the insert(s) to determine if proper correction is achieved or if a correction is deemed to not be adequate by the surgeon. If an error is made or if additional adjustments need to be made, the insert(s) can be removed and replaced with other insert(s) to adjust the implant offset relative to the bone so that the implant will be properly positioned relative to the bone and the revised implant offset can be trialed again. The trialing can be passed when the surgeon determines that the insert offset will provide a sufficient stability and range of motion. The trialing requirements can be predetermined. However, in some embodiments, the surgeon may need to determine a best fit insert which will provide the best surgical outcome for the patient based upon empirical trial and error rather than strict offset measurements. This insert replacement and trialing process can be repeated until the inserts that properly position the implant are found and the trial assessment is passed. (See add in notes to put in this general area. Test for rotation, Too much laxity then need to tighten need to add bigger femur Tighter in flexion.)

In an embodiment, once the correct offset is achieved as determined by the surgeon, the cured PMMA inserts can be left in place in or on the bone. Liquid PMMA cement can be applied to bond the inserts and the bone. The final bone implant can be unwrapped and placed on the liquid PMMA, inserts and bone. The liquid PMMA cement will then cure to chemically bond to the PMMA inserts and mechanically bond the implant to the bone. The cured PMMA cement and the PMMA inserts can form a solid substantially homogeneous high strength structure between the implant and bone. The insert offsets may only be applied to the bony surfaces and the final implant is not altered in any way, which improves the efficiency of the arthroplasty.

As discussed, various different types of inserts can be used to offset the implant such as: tacks, shims and/or rods. A tack insert can include a stem that is in direct physical contact with a cap. In a tack embodiment, the stem is inserted into the bone and a bottom surface of the cap adjacent to the stem can contact the bone surface and the thickness of the cap can provide a predetermined offset. A trial implant can be placed in contact with the top surface of the cap opposite the stem and trialing of the insert offsets can be performed. Different tacks having different cap thicknesses can be available to change the implant offset from the bone and if adjustments are necessary the tack inserts can be replaced and trialed. The tacks can also have tapered or angled caps which are not uniform in thickness. For example, the upper and lower surface of the cap can include non-parallel planes and the intersection of the planes can define an acute angle. Once the tack inserts that provide the proper implant offset to pass the trialing are found the liquid PMMA cement can be used to create a chemical bond with the PMMA inserts and mechanically bond the final implant to the bone.

In different embodiments, the inventive system can be used for bone deficiency issues with cured PMMA inserts being used for augmentation of liquid PMMA cement. In an embodiment, pre-cured PMMA insert structures can pre-penetrate bony surfaces. For example, stemmed augments can be inserted into holes formed in bones which can function like strengthening PMMA rebar in liquid PMMA cement. The technique that utilize the procured PMMA structures can include: 1) placing PMMA inserts into bone and across bony surfaces, 2) applying cement to surface(s) of the bone and implant, 3) applying implant to the bone interface, and 4) curing the PMMA cement to create a chemical bond with the PMMA inserts and a mechanical bond between the implant and the host bone. The entire assembly of bone, PMMA inserts, PMMA cement and the implant can be a composite structure.

In an embodiment, the inserts can be cured PMMA rods which can be inserted into a host bone. A portion of the rods can extend away from the bony surfaces. Distal ends of the PMMA rods may rest against the bottoms of the holes formed in the bone. The offsets of the rods extending from the holes can be controlled by the lengths of the rods and the depths of the holes. The offset of the implant can be tested against the ends of the rods with a trial implant and an assessment can be made of the rod inserts. If offset adjustments need to be made, the rods can be replaced with different length rods. In some embodiments, if the rods need to be shortened, they can be cut or broken. In other embodiments, the rods may not contact the implant. Once the proper PMMA rods have been inserted into the bone, liquid PMMA cement can be applied to the bone, inserts and implant. The liquid PMMA cement can cure to form a chemical bond with the PMMA rods and a mechanical bond between the implant and the host bone.

In a PMMA shim insert embodiment, the insert structure can include a head having a larger cross section and a shape that can correspond to features of the implant. The surgeon can have a set of shims which can have different thicknesses and the upper and lower surface of the heads of the shims can be non-parallel planes and the intersection of the planes can define an acute angle. The shims can include one or more stems which are inserted into the bone. The surgeon can perform a trial assessment with a trial implant to determine if the inserts will provide the proper implant offset. If there is an error in the offset of the implant relative to the bone, the shims can be removed and replaced with shims having different thicknesses and/or angles. Once a set of shims passes the trial assessment, liquid PMMA can be applied to the bone, inserts and implant. The liquid PMMA cement can cure and chemically bond with the PMMA inserts and form a strong mechanical bond between the implant and the host bone.

Although the cured PMMA inserts are described as being made of PMMA, in some embodiments, the inserts can include other materials. For example, the inserts may include metal or polymer substrates. For example, a metal rod such as stainless steel or titanium can be encapsulated within the cured PMMA in the stem area, or alternatively, an entire metal tack can be encapsulated within cured PMMA. In other embodiments, the inserts can include polymer structures encapsulated with cured PMMA. This composite design can be useful when higher structural strength is needed for the PMMA implant which can increase the mechanical properties such as shear and compression strength.

The inventive system can be particularly useful in surgeries that require a very high implant position accuracy such as Total Knee Arthroplasty (TKA). In an embodiment, a TKA may require resectioning the distal end of the femur. If there are any errors in the resection surfaces the femoral implant will not be positioned correctly relative to the femur. The error may cause the patient to have difficulty walking because the length and/or angle of the reconstructed limb may be incorrect. Incorrect balancing of the knee is associated with increased pain and inflammation and decreased function. In an embodiment, the TKR may include a first resection surface on a medial condyle of the femur (MFC) and the second resection surface on a lateral femoral condyle of the femur (LFC). If there are errors in the MFC and/or LFC resection surfaces, inserts placed on these surfaces can be used to correct the position of the implant relative to the bone. More specifically, medial and lateral inserts having the same offsets can be used if the errors are only in length or distal offset and there are no angular errors. In contrast, medial and lateral inserts having different offsets can be used if there are angular and length offset errors or a single insert can be used to make angular correction. This invention describes an efficient technique for using corrective PMMA inserts that are stable for trial reductions and stress testing and require no additional manipulation is required once the trial reduction determines that the positioning of an implant bonded to the inserts is correct.

In different embodiments, tack inserts can be used with a distal femoral application. For example, in different scenarios the inventive system can be used for femoral over-resections where there is a flexion/extension mismatch. The inventive system can provide corrective options. The inventive system can be used to lower a joint line which can quickly and accurately make alignment adjustments so that any alignment imbalance can be corrected. In some embodiments, the inventive system can be used with tacks placed in multiple surfaces of the bone such as the anterior, posterior and/or distal surface(s) when the position of the implant needs to be adjusted.

In an embodiment, the PMMA inserts can be used to make angular corrections such as angular augments and asymmetric augments. In the illustration, the head of the insert or a shim structure between the bone and the implant can be angled. The thickness of the shim can be thicker at one side surface than the opposite side. This can allow the angle between the implant and the bone to be adjusted. This angular correction can be used for various angular scenarios including: restoring the correct angulation if there is over-resection. The PMMA inserts can be applied to bony surfaces and the different thicknesses to create different angular corrections. These angular corrections can be applied to single or multiple surfaces of the bone.

### BRIEF DESCRIPTION OF THE DRAWINGS

FIG. 1 illustrates a side view of a bone;

FIG. 2 illustrates a side view of a bone with resectioned surfaces;

FIG. 3 illustrates a side view of a bone with an embodiment of a PMMA tack insert in a distal resection surface;

FIG. 4 illustrates a side view of an implant bonded to a bone and a tack on a PMMA distal resection surface;

FIG. 5 illustrates a side of view of a bone with an embodiment of a PMMA tack insert in an anterior resection surface;

FIG. 6 illustrates a side view of an implant bonded to a bone and a PMMA tack insert on an anterior resection surface;

FIG. 7 illustrates an anterior view of a femur and tibia;

FIG. 8 illustrates an anterior view of a distal portion of a femur;

FIG. 9 illustrates an anterior view of a femur with a distal resection surface with PMMA tack inserts;

FIG. 10 illustrates an anterior view of a femur with a distal resection surface with PMMA tack inserts with a trial implant;

FIG. 11 illustrates an anterior view of a femur with a distal resection surface with PMMA tack inserts bonded to a final implant;

FIG. 12 illustrates an anterior view of a femur with a distal resection surface with PMMA tack inserts and a trial implant;

FIG. 13 illustrates an anterior view of a femur with a distal resection surface with PMMA tack inserts bonded to a final implant;

FIG. 14 illustrates an anterior view of a femur with a distal resection surface with PMMA tack inserts and a trial implant;

FIG. 15 illustrates an anterior view of a femur with a distal resection surface with PMMA tack inserts bonded to a final implant;

FIG. 16 illustrates an anterior view of a femur with a distal resection surface with a trial implant;

FIG. 17 illustrates an anterior view of a femur with a distal resection surface with a PMMA tack insert bonded to a final implant;

FIG. 18 illustrates an anterior view of a femur with a distal resection surface with a trial implant;

FIG. 19 illustrates an anterior view of a femur with a distal resection surface with a PMMA tack insert bonded to a final implant;

FIG. 20 illustrates a side view of a bone with PMMA tack inserts in anterior and distal resection surfaces;

FIG. 21 illustrates a side view of a bone with PMMA tack inserts in anterior and distal resection surfaces bonded to a final insert;

FIG. 22 illustrates a side view of a bone with a PMMA tack insert in a posterior resection surface;

FIG. 23 illustrates a side view of a bone with a PMMA tack insert in a posterior resection surface bonded to a final insert;

FIGS. 24-26 illustrate flow charts for adjusting PMMA inserts and bonding a final implant to a bone;

FIGS. 27-35 illustrate side views of different embodiments of PMMA tack inserts;

FIG. 36 illustrates a top view of an embodiment of a PMMA tack insert;

FIG. 37 illustrates a side view of an embodiment of a PMMA tack insert in a bone;

FIGS. 38-40 illustrate side views of different embodiments of PMMA tack inserts with bone retention mechanisms;

FIG. 41 illustrates a top view of an embodiment of a PMMA tack insert;

FIG. 42 illustrates a side view of an embodiment of a PMMA tack insert;

FIGS. 43 and 44 illustrate a bottom views of an embodiment of the stem of a PMMA tack insert;

FIG. 45 illustrates a side view of a canulated embodiment of a PMMA tack insert;

FIG. 46 illustrates a bottom view of a canulated embodiment of a PMMA tack insert;

FIG. 47 illustrates a side view of a PMMA rod insert;

FIG. 48 illustrates a side view of a resectioned bone with PMMA rod inserts;

FIG. 49 illustrates a side view of a resectioned bone with PMMA rod inserts and liquid PMMA cement;

FIGS. 50 and 51 illustrate side views of resectioned bones with PMMA rod inserts and PMMA cement bonded to final implants;

FIGS. 52 and 53 illustrate side views of resectioned bones with PMMA rod inserts;

FIG. 54 illustrates a side view of a resectioned bone with PMMA rod inserts and liquid PMMA cement;

FIG. 55 illustrates a side view of a resectioned bone with PMMA rod inserts and liquid PMMA cement bonded to a final implant.

FIG. 56 illustrates a bone with PMMA rod inserts inserted into internal surfaces;

FIG. 57 illustrates a bone with PMMA rod inserts in internal bone surfaces and liquid PMMA cement;

FIG. 58 illustrates a bone with PMMA rod inserts in internal bone surfaces and a final implant inserted into the liquid PMMA cement;

FIG. 59 illustrates a bone with PMMA rod inserts in internal bone surfaces and a final implant bonded to the bone with the liquid PMMA cement;

FIG. 60 illustrates a flow chart of for adjusting PMMA rod inserts and bonding a final implant to a bone.

FIG. 61 illustrates a side view of a PMMA rod insert with fracture lines;

FIG. 62 illustrates a side view of a PMMA rod insert in a bone hole;

FIGS. 63 and 64 illustrate side views of a shortened PMMA rod insert in a bone hole;

FIG. 65 illustrates a bone with a shim on anterior and distal surfaces;

FIG. 66 illustrates a bone with a shim on anterior and distal surfaces bonded to a final implant;

FIG. 67 illustrates a bone with a shim on anterior, distal and posterior surfaces;

FIG. 68 illustrates a bone with a shim on anterior, distal and posterior surfaces bonded to a final implant;

FIG. 69 illustrates a bone with a shim on anterior and distal surfaces;

FIG. 70 illustrates a bone with a shim on anterior and distal surfaces bonded to a final implant;

FIG. 71 illustrates a bone with a shim on anterior, distal and posterior surfaces;

FIG. 72 illustrates a bone with a shim on anterior, distal and posterior surfaces bonded to a final implant;

FIG. 73 illustrates an anterior view of a bone with PMMA shim inserts with a trial implant;

FIG. 74 illustrates an anterior view of a bone with PMMA shim inserts with a final implant;

FIG. 75 illustrates an anterior view of a bone with PMMA shim inserts with a trial implant;

FIG. 76 illustrates an anterior view of a bone with PMMA shim inserts with a final implant;

FIGS. 77-84 illustrate perspective views of different embodiments of PMMA shims;

FIG. 85 illustrates a side view of an embodiment of a bone drill;

FIG. 86 illustrates an top view of an embodiment of a bone drill;

FIG. 87 illustrates a set of PMMA tack inserts used in an augmentation kit;

FIG. 88 illustrates a modular PMMA tack with separated PMMA cap attachments;

FIG. 89 illustrates a modular PMMA tack coupled to PMMA cap attachments;

FIG. 90 illustrates a modular PMMA tack coupled to PMMA cap attachments in a bone hole;

FIG. 91 illustrates a top view of a tool used with the PMMA cap attachments;

FIG. 92 illustrates a side view of an embodiment of a threaded PMMA insert;

FIGS. 93-94 illustrate side views of an embodiment of a threaded PMMA insert positioned at different offsets in a bone;

FIGS. 95-96 illustrate side views of an embodiment of a threaded PMMA insert rotated by an insertion tool in a bone;

FIG. 97 illustrates a top view of a PMMA spacer;

FIG. 98 illustrates a perspective view of a PMMA spacer;

FIG. 99 illustrates a side view of an embodiment of a threaded PMMA insert and a PMMA spacer;

FIG. 100 illustrates a side view of an embodiment of a threaded PMMA insert and multiple PMMA spacers;

FIG. 101 illustrates a side view of a threaded and expandable PMMA insert in a bone;

FIGS. 102-103 illustrate side views of a threaded and expandable PMMA insert in a bone with an expansion screw;

FIGS. 104-106 illustrate side views of embodiments of modular composite PMMA tack inserts.

FIG. 107 illustrates a perspective view of a patella bone with a patella implant;

FIG. 108 illustrates a perspective view of a patella bone with a tack insert and a patella implant;

FIG. 109 illustrates a side view of a patella bone with a tack insert and a patella implant;

FIG. 110 illustrates a bottom view of a distal end of a bone;

FIGS. 111-112 illustrate bottom views of a distal end of a bone after resectioning;

FIG. 113 illustrates a bottom view of a distal end of a bone with tack inserts.

FIG. 114 illustrates a side view of a tack insert having two layer cap; and

FIG. 115 illustrates a view of a bone with a resection surface with PMMA tack inserts bonded to a final implant.

### DETAILED DESCRIPTION

The present invention is directed towards bone implant augment methods and apparatus for surgical procedures such as Total Knee Arthroplasty (TKA). The present invention can allow surgeons to meet increased technical demands and expectations of both speed and accuracy. The inventive systems can provide more accurate adjustments to implant positions to supplement existing instrumentation and smaller increment implant sizing which can improve the accuracy limitations of mechanical systems due to imperfect mechanical resection bone cuts in both axial length and angular orientation.

The present invention can provide a system and method for adjusting the bone implant components for these imperfect mechanical bone cuts. The inventive devices for correction of imperfect resection cuts are accurate and quickly installed during surgery so that the offset and position of the implant relative to the bone is corrected. The inventive system and apparatus can minimize surgical delays and can be provided in a simple surgical kit. The kit components can include cured PMMA inserts which can integrate with existing techniques/methods and tools. Once installed in the patient there may not be any radiographic evidence and no adverse impact on mechanical integrity of the final construct. In situations with hardened or sclerotic bone, the final bone implant construct of precured PMMA inserts within the bone and chemically bonded to the cured liquid cement has stronger mechanical properties at the bone PMMA cement interface than an implant mechanically bonded to a bone with just PMMA cement. In some embodiments, the inserts can have high strength substrates which are encapsulated in cured PMMA which can improve the strength of the implant connection to the bone.

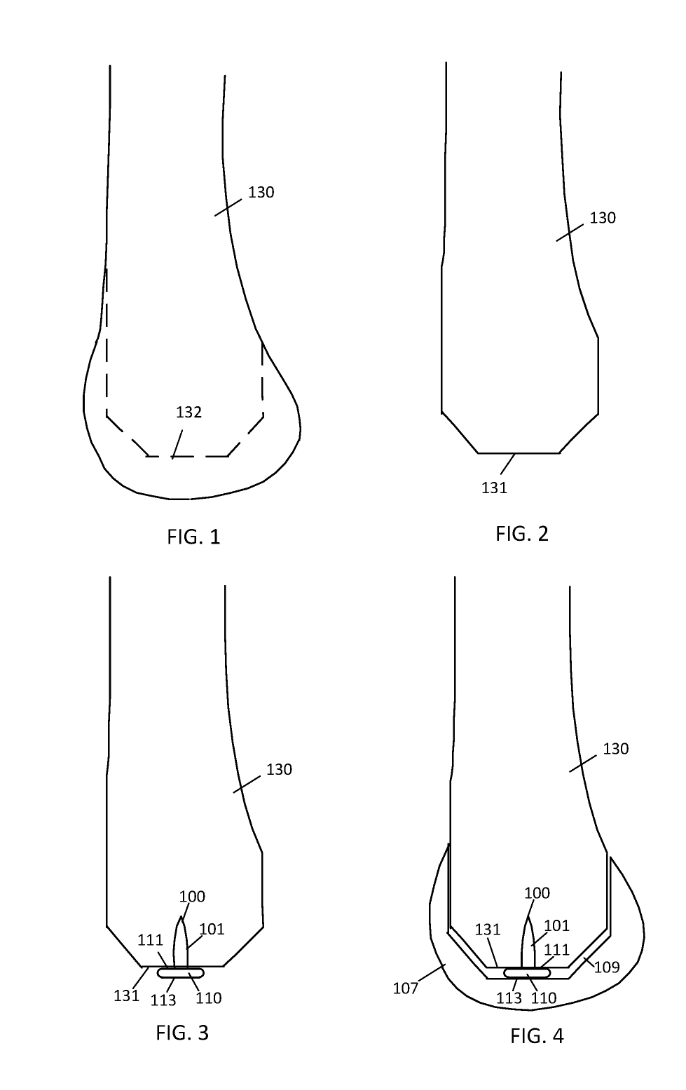

FIGS. 1-4 illustrates side views of a femur bone 130 to which a bone implant 107 will be bonded to. With reference to FIG. 1, a bone 130 is illustrated with markings 132 indicating locations of resection cuts. With reference to FIG. 2, the bone 130 has been cut and includes resection surfaces 131. With reference to FIG. 3, a cured PMMA insert 100 has been placed in the resection surface 131. In this embodiment, the cured PMMA insert 100 includes a cap 110 and a stem 101. The cured PMMA insert 100 has been fully inserted into the bone 130 so that a first surface 111 of the cap 110 is adjacent to and in direct physical contact with the resection surface 131. With reference to FIG. 4, the implant 107 is placed on the bone 130 with a surface of the implant 107 in direct physical contact with a second surface 113 of the cap 110 which is opposite the first surface 111.

Liquid PMMA cement 109 can be applied to the bone 130, insert 100 and implant 107. In an embodiment, the liquid PMMA cement 109 can be pressurized and injected into the space between the implant 107 and the bone 130. The liquid PMMA cement 109 can cure and chemically bond to the PMMA insert 100 and create a strong mechanical bond between the implant 107 and the bone 130. In an embodiment, the internal surfaces of the implant 107 can be coated with materials that can chemically bond to the liquid PMMA cement 109. For example, the inner surfaces of the implant can be coated with cured PMMA. In other embodiments, the interior surfaces of the implant 107 can be textured or have physical features such as grooves, holes, fenestrations, etc. which can improve the interdigitation of the liquid PMMA cement with the implant 107.

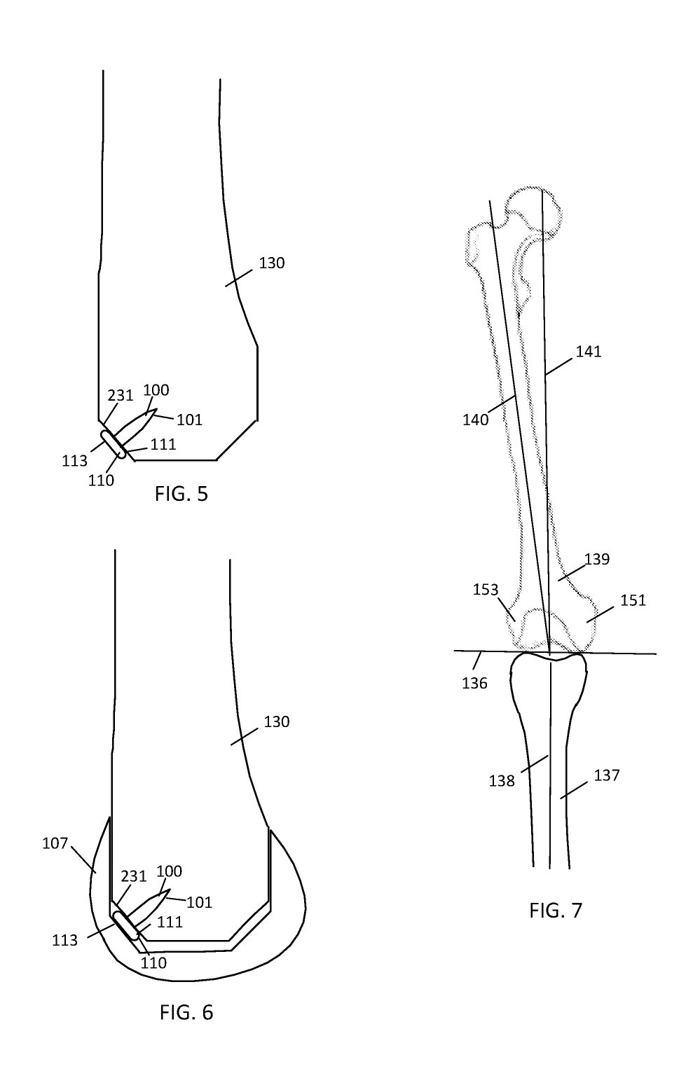

In other embodiments, the cured PMMA insert 100 can be inserted into a different resection surface such as an anterior resection surface With reference to FIG. 5, a cured PMMA insert 100 has been placed in the anterior resection surface 231. In this embodiment, the cured PMMA insert 100 includes a cap 110 and a stem 101. The cured PMMA insert 100 has been fully inserted into the bone 130 so that a first surface 111 of the cap 110 is adjacent to and in direct physical contact with the anterior chamfer resection surface 231. With reference to FIG. 6, the implant 107 is placed on the bone 130 with a surface of the implant 107 in direct physical contact with a second surface 113 of the cap 110 which is opposite the first surface 111. In other embodiments, the cured PMMA insert 100 can be placed on any surface of the bone 130 between the bone 130 and the implant 107.

The insertion of the cured PMMA insert 100 into the bone 130 can comprise various procedural steps. In an embodiment, the bone resection surface can be drilled and the insert 100 can be placed into the hole formed. The drill can be a stepped drill bit which creates an insert hole having a specific depth and diameter. In other embodiments, the cured PMMA insert 100 can be physically pressed into the bone 130. The force of the stem 101 against the bone 130 can create the hole in the bone. The surgeon can then trial the offset of the insert 100 to determine the proper offset of the insert 100. If the insert needs to be replaced, the insert 100 can be removed and a replacement insert 100 can be pressed into the same hole formed by the previously trialed insert 100. The cured PMMA inserts 100 can have caps 110 that have structural features that can allow the surgeon to easily remove the cured PMMA inserts 100. In the illustrated example, the caps 110 can have a rounded outer surface facing the bone 130 that allows the surgeon to grasp and pull up on the cap 100. In other embodiments, a tool can be used to grasp and/or pull the cap 110 away from the bone 130.

The inventive process solves a significant problem that occurs when too much bone is removed during resectioning. There are no known methods for easily adding bone material to the cut bone surfaces and readjusting the bone to compensate for over cuts can be impossible. The application of cured PMMA inserts solves this problem by allowing surgeons to increase the implant offset and has the added benefit of providing a stronger bond between the bone and implant because the implants can be secured to stems mechanically bonded in holes in the bone. In contrast, a normal bone implant may only rely upon PMMA cement placed on the outer surfaces of the bone to provide the mechanical bonding to the implant.

The alignment of the implant can be based upon the anatomical axis of the patient rather than a mechanical axis. With reference to FIG. 7, an anterior view of the knee joint is illustrated. The distal surfaces of the femur 137 can be a horizontal axis that is parallel to the rotational axis of the knee 136. Each patient's anatomical geometry can be different and the femur 139 can have various alignment configurations with the tibia 138. In the illustrated example, the geometric axis 141 of the tibia 138 can be defined by a line between the head at the proximal end of the femur 139 and the center of the knee. The geometric axis 141 can be perpendicular to the rotational axis of the knee 136 and aligned with the center axis of the tibia 138. As illustrated, the anatomic center axis 140 of the femur 139 is angled from the geometric center axis 141 of the tibia 138 and is not be perpendicular to the rotational axis of the knee 136 in the illustrated example. However, in other embodiments (not illustrated) the surgeon may configure the patient's leg with the anatomical axis 140 of the femur 139 in a perpendicular orientation relative to the rotational axis 136 of the knee and aligned with the center axis 138 of the tibia 137.

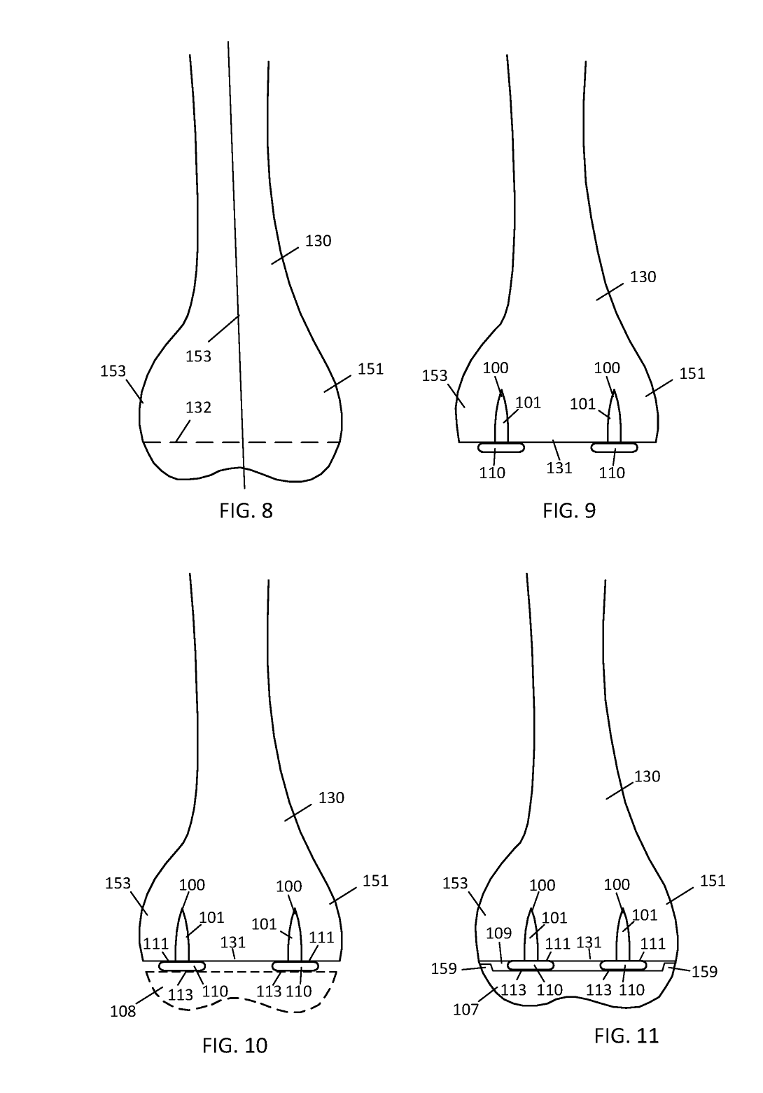

FIGS. 8-11 illustrate anterior view of a femur bone 130 and bone implant 107. With reference to FIG. 8, the bone 130 is illustrated with a lateral condyle of the femur (LFC) 153 and a medial condyle of the femur (MFC) 151. The resection cut markings 132 extends through portions of both the lateral condyle 153 and the medial condyle 151. The resection cut markings 132 may not be perpendicular to the center axis of the femur 153. FIG. 9 illustrates the bone 130 after being cut with a resection surface 131 and with cured PMMA inserts 100 placed in the resection surface 131 on the LFC 153 and MFC 151. The cured PMMA inserts 100 have been fully inserted into the bone 130 so that a first surface 111 of the caps 110 are in direct physical contact with the resection surface 131 of the bone 130. The implant 107 is placed on the bone 130 and in direct physical contact with the second surfaces 113 of the caps 110.

With reference to FIG. 10, the surgeon can check the offset of the implant 107 relative to the bone 130 and determine if the offset is correct. Checking the offset can include length and angular offset measurements of the implant 107 relative to the bone 130. Checking can also be performed for functional performance with use of trial implants and range of motion of the joint with assessment of stability and motion. If changes need to be made, the cured PMMA inserts 100 can be removed and replaced with another insert that has a cap 110 having a different thickness or a different angle between the first surfaces 111 and the second surfaces 113. The position of the implant can be checked with a trial implant 108 and various mechanical tests can be performed to determine if the implant will be properly positioned by the inserts 100. With reference to FIG. 11, once the proper inserts are found to properly position the implant, liquid PMMA 109 can be applied to the inserts 100, resection surface 131 of the bone 130 and the implant 107. The liquid PMMA can cure to bond the implant 107 to the bone 130 and inserts 100. In the illustrated embodiment, the implant 107 can include a raised edge 159 which can extend around the outer perimeter of the implant 107. The raised edge 159 can function to help retain the liquid PMMA cement 109 within the space between the bone 130 and the implant 107. The height of the raised edge 159 can be less than the thickness of the insert 100 so that the implant 107 will contact the inserts 100 but the raised edge 159 will not contact the bone 130.

In some embodiments, the raised edge of the implant can engage features of the tack inserts. For example, with reference to FIG. 114 an embodiment of a cured PMMA tack insert 601 is illustrated which has a stem 101 and a stepped cap with a lower cap 605 and an upper cap 603. In the illustrated embodiment, the lower cap 605 can have a smaller outer diameter than the upper cap 603. In other embodiments, the lower cap 605 can have an exposed surface that is not covered by the upper cap 603. With reference to FIG. 115, as discussed above with reference to FIG. 11, the implant 107 can have a raised edge 159 which can be on an edge of the implant 107 bonding surface 609. In an embodiment, the stems 101 of the PMMA tack inserts 601 can be pressed into a resection surface 131 of the bone 130. The tack inserts 601 can be trialed with a trial implant. If the tack inserts 601 provide the proper implant 107 offset, liquid PMMA cement 109 can be applied to the bonding surface 609, the internal raised edge 159 surfaces, the tack insert 601 and the resection surface 131 of the bone 130. The implant 107 can be placed on the tack inserts 601 with the raised edges 159 adjacent to the lower caps 605 and the bonding surface 609 adjacent to the upper caps 603. In an embodiment, the offset of the upper cap 603 from the lower cap 605 can be the same or similar to the height of the raised edge 159 from the bonding surface 609. Be raised edge 159 can also be place in close proximity to the outer side surfaces of the upper caps 603. Thus, the raised caps 603 can function as indexes to help place the implant 107 in the proper aligned position on the resection surface 131 of the bone 130.

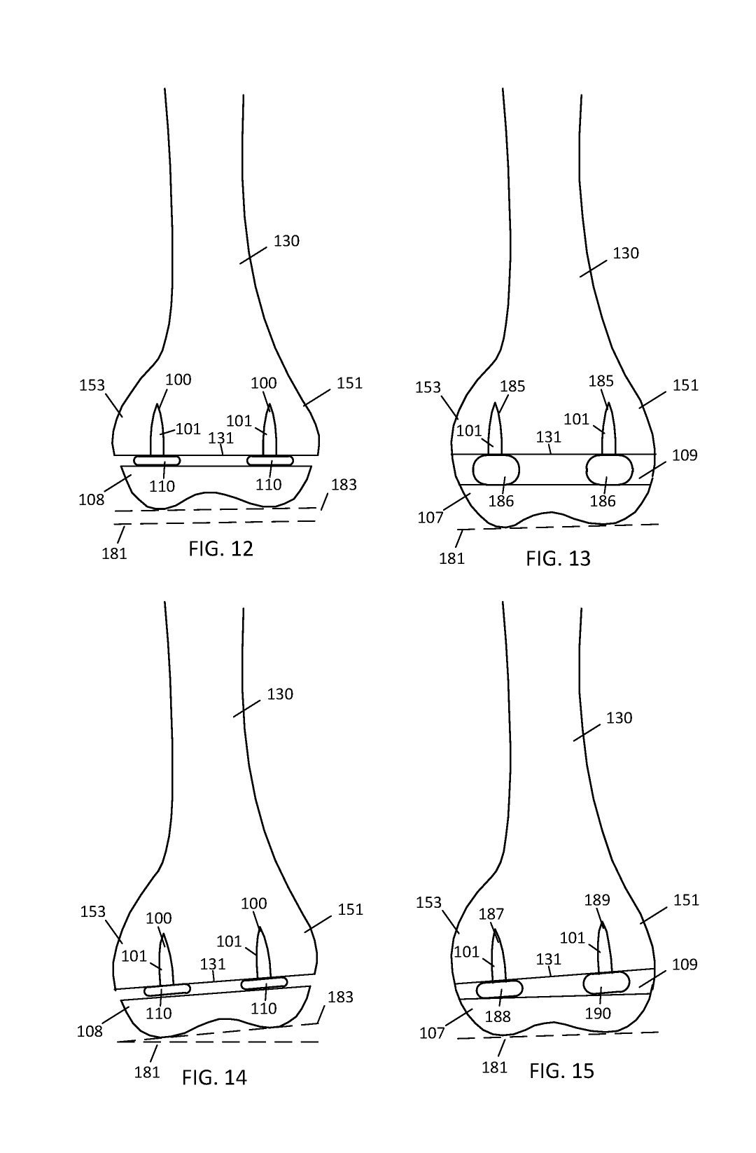

FIGS. 12-15 illustrate adjusting the cap thicknesses of the inserts to properly offset the implant. With reference to FIG. 12, the proper predetermined length offset of the implant 107 relative to the bone 130 can be represented by line 181. However, in the illustrated embodiment, the measured, calculated or trialed with a trial implant 108 to determine that the offset line 183 is substantially shorter than the proper offset line 181. The offset line 183 can be determined during a trial process of the inserts 100 where a trial implant is placed on the inserts 100 and the stability and range of motion can be tested. If these trial tests fail, the surgeon can make corrective adjustments to the inserts 100 to alter the offset so the final implant will match the offset line 183. With reference to FIG. 13, the length of the offset between the bone 130 and the implant 107 has been altered by replacing the inserts 100 with replacement inserts 185 having thicker caps 186. With the replacement inserts 185, the offset of the final implant 107 matches the proper predetermined length offset line 181. If the offset position of the implant needs to be shortened, the inserts can be replaced with inserts having thinner caps. In this embodiment, the angle of the resection surface 131 was correct, so the thicker caps 186 of the inserts 185 can have the same thickness so that the angle of the implant 107 is not changed relative to the bone 130. Liquid PMMA 109 can be applied to the bone 130, inserts 185 and final implant 107. The liquid PMMA 109 can cure to chemically bond to the PMMA inserts 183 and mechanically bond the implant 107 to the bone 130.

With reference to FIG. 14, an embodiment is illustrated where the inserts 100 and the measured, calculated or determined offset line 183 is at a different angle than the proper offset line 181 during trialing. The offset angle of the trial implant 108 relative to the bone 130 can be changed and corrected by using inserts 100 having different thickness caps 110. With reference to FIG. 15, the original inserts 100 have been removed and replaced with a first insert 187 which has a thick cap 188 in the LFC and a second insert 189 which has a thicker cap 190 in the MFC. These replacement inserts 187, 189 can cause the final implant 107 offset to be properly angled and positioned and match the correct predetermined offset line 181. FIGS. 14 and 15 illustrate one embodiment of an angular correction. However, if the surgeon needs to angle the implant 107 more towards the medial side, the insert 100 placed in the MFC 151 can have a thinner cap 110 than the cap 110 on the insert 100 placed in the LFC 153.

With reference to FIGS. 13 and 15, once the surgeon determines that the inserts 100 will provide the proper offset of the implant 107 relative to the bone 130 by a trial process, a liquid PMMA cement 109 can be applied to the contact and non-contact surfaces of the insert 100, the resection surface 131 of the bone 130 and the implant 107. The liquid PMMA cement 109 may also injected or placed in the spaces between the bone 130 and the implant 107 around the cap 110. The liquid PMMA cement 109 cannot be placed in areas that are not between the bone 130 and the implant 107. The liquid PMMA cement will harden into a solid and chemically bond to the cured PMMA insert 100 and mechanically bond the bone 130 to the implant 107. Once cured and fully hardened, the implant 107 will be rigidly attached to the bone 130.

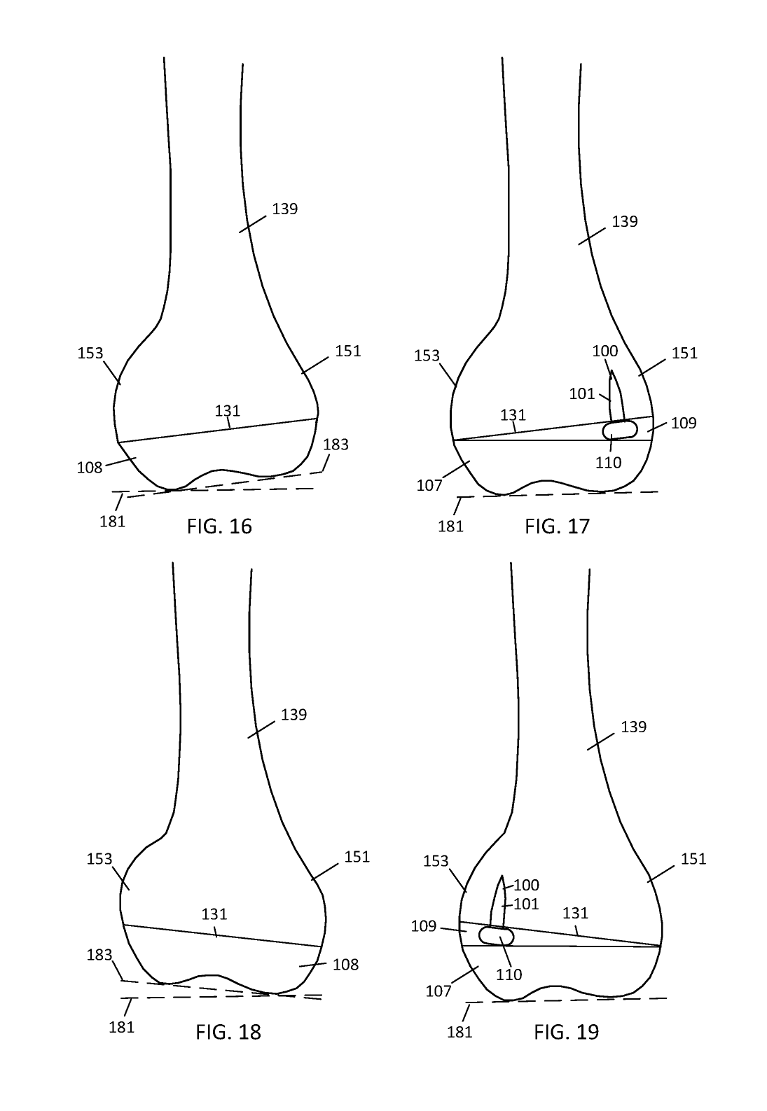

FIGS. 11-15 illustrate embodiments where two inserts are used to make corrections to the implant offset relative to the femur. However, in other embodiments is can be possible to make angular corrections to the offset of the implant with a single implant. FIG. 16 illustrates a femur 139 with a resection surface 131. A trial implant 108 can be placed on the resection surface 131 and the surgeon can perform a trial process and determine that the measured offset line 183 does not match the correct offset line 181 and material needs to be added to the MFC 151 side of the resection surface 131. With reference to FIG. 17, a stem 101 of the tack insert 100 is inserted into the MFC 151 side of the resection surface 131 and the trial process can be repeated. If the trial process is passed, liquid PMMA 109 can be applied to the resection surface 131, insert 100 and the implant 107 to mechanically bond the implant 107 to the femur 139. With reference to FIG. 18, a trial implant 108 is attached to the resection surface 131 and the trial process can determine that material needs to be added to the LFC 153 side of the resection surface. With reference to FIG. 19, a tack insert 100 is inserted into the LFC 153 side of the resection surface 131 to correct the offset of the implant 107. When the trial testing has been passed, liquid PMMA 109 can be applied to the resection surface 131, insert 100 and the implant 107 to mechanically bond the implant 107 to the femur 139.

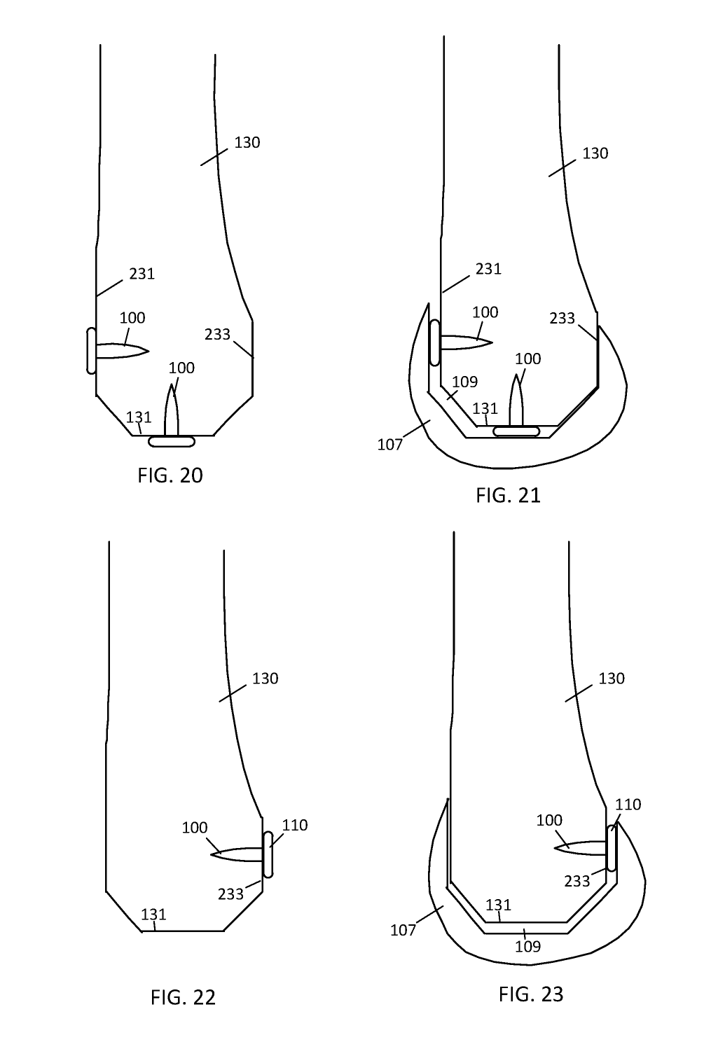

With reference to FIGS. 20-23, side views of a bone 130 having multiple resection surfaces 131 are illustrated. In some embodiments, the inserts 100 can be placed on multiple resection surfaces 131 which are not in the same plane. The inserts 100 can allow the surgeon to move the implant 107 towards the anterior or posterior sides of the bone 130. With reference to FIG. 20, one or more inserts 100 are placed in an anterior resection surface 231 and a distal surface 131 that can be perpendicular to a center axis of the bone 130 With reference to FIG. 21, the implant 107 position relative to the bone 130 can be adjusted towards the anterior surface by placing inserts 100 in an anterior resection surface 231. With reference to FIG. 22, one or more inserts 100 are placed in a posterior resection surface 233 that can be substantially parallel to a center axis of the bone 130. With reference to FIG. 23, the implant 107 is moved towards the posterior surface relative to the bone 130 by using inserts 100 that have different offsets in the posterior surfaces 233. By having inserts 100 in multiple resection surfaces the surgeon can have more precise control of the position of the implant 107 relative to the bone 130 to match the predetermined required offset distances, relative positions and angles in three dimensional space. Placement of implants 100 in the posterior resection surface 233 will allow the surgeon to securely increase the size of a femoral component to reduce a selective flexion gap imbalance.

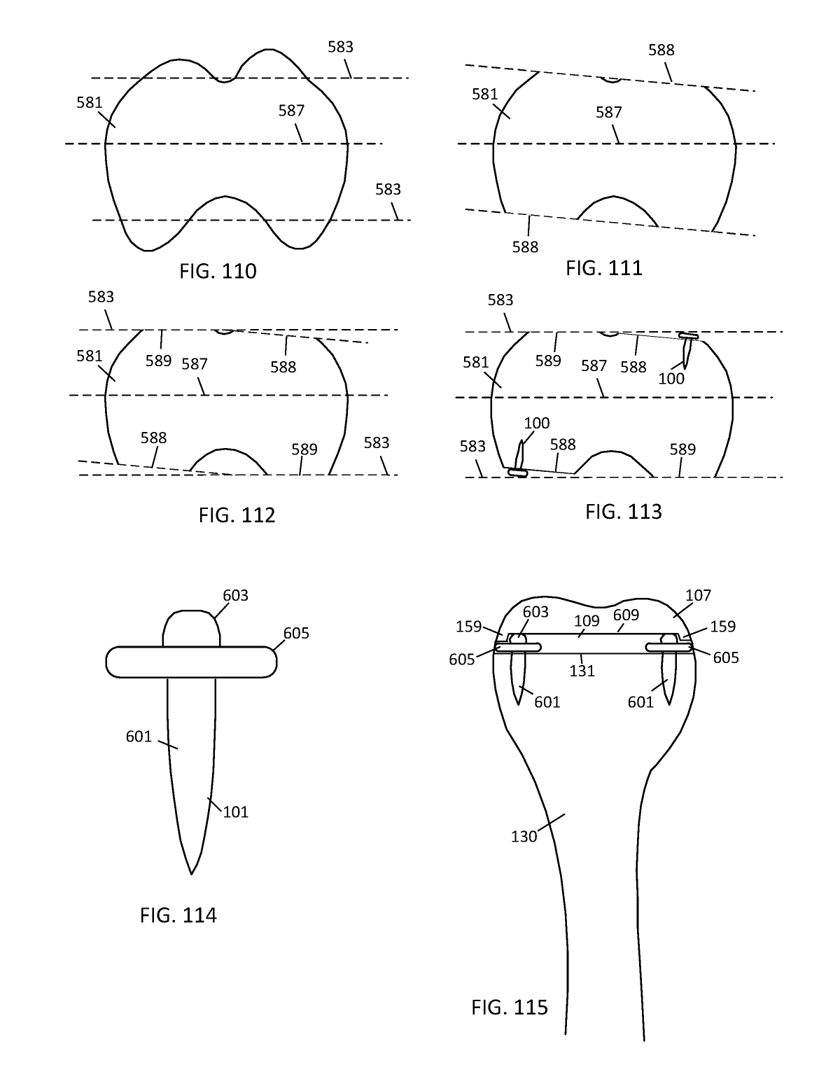

The present invention illustrates how an implant can be offset relative to a bone in different directions in three-dimensional space. In an embodiment, the bone can be aligned with an X, Y, and Z coordinate system with the center axis of the bone aligned with the Z-axis. The anterior surface can face the X-axis and the joint at the distal end of the bone can rotate about the Y-axis. With reference to an X, Y, Z coordinate system, FIGS. 9-13 illustrate how cured PMMA inserts can be used to offset the implant from the bone in the Z direction and FIGS. 20-23 can illustrate how PMMA inserts can be used to offset the implant from the bone in the X direction. In these illustrations the implant can have a “U” shape so that the surgeon can move the implant manually in the Y direction. The cured PMMA inserts can used to control the rotation of the implant relative to the bone about the X axis, Y axis and Z axis. More specifically, FIGS. 16-19 illustrate how the PMMA inserts are used to adjust the rotation of the implant about the X-axis. Similarly, the PMMA inserts illustrated in FIGS. 20-23 can be used to control the rotation of the implant about the Y-axis. FIGS. 110-113 illustrate how the cured PMMA inserts can be used to control the offset rotation of the implant about the Z-axis.

FIG. 110 illustrates a bottom view of an embodiment of a bone 581 that can be marked with resection lines 583 which indicate the portions of the bone 581 that will be cut. The implant can be attached to the bone 581 at resection surfaces and the resection lines 583 can be parallel to a joint line 585 which can define an axis of rotation 587. With reference to FIG. 111, an error can be made during resectioning of the bone 581 and the resection surfaces 588 may not be parallel to the axis of rotation 587. Attaching the implant to this defective resection surfaces 588 would result in misalignment of the implant about the Z-axis relative to the bone 581. With reference to FIG. 112, in order to correct this problem, an additional resection cut or cuts 588 may be necessary. However, once the additional resection cuts 589 are made, the resection surfaces 588 may need to be built up to provide a proper planar surface for positioning the implant in a correction position and rotation relative to the bone. With reference to FIG. 113, tack inserts 100 can be placed in the resection surfaces 588 of the bone 581 can provide the proper Z axis rotation offset so that the implant can be parallel to the axis of rotation 587. As discussed, trialing can be performed on the inserts 100 using a trail implant until proper offset inserts are found. Once the properly sized inserts are found, liquid PMMA can be applied to the bone, PMMA inserts and implant. The implant can be placed against the bone and PMMA inserts. The liquid PMMA can cure forming a chemical bond with the PMMA inserts and form a mechanical bond between the bone 581 and the implant.

In embodiments, the cured PMMA inserts 100 and/or the liquid PMMA cement 109 may have a radiopaque additive which can be detected by x-rays. The bone implant and bone can be x-rayed to determine if the cured PMMA inserts 100 are properly positioned in the bone 130 and determine if the liquid PMMA cement has been properly placed on all of the required surfaces and spaces between the bone 130 and the implant 107 to insure the implant 107 will be properly bonded to the bone 130. If errors are detected, additional PMMA cement 109 can be applied where needed.

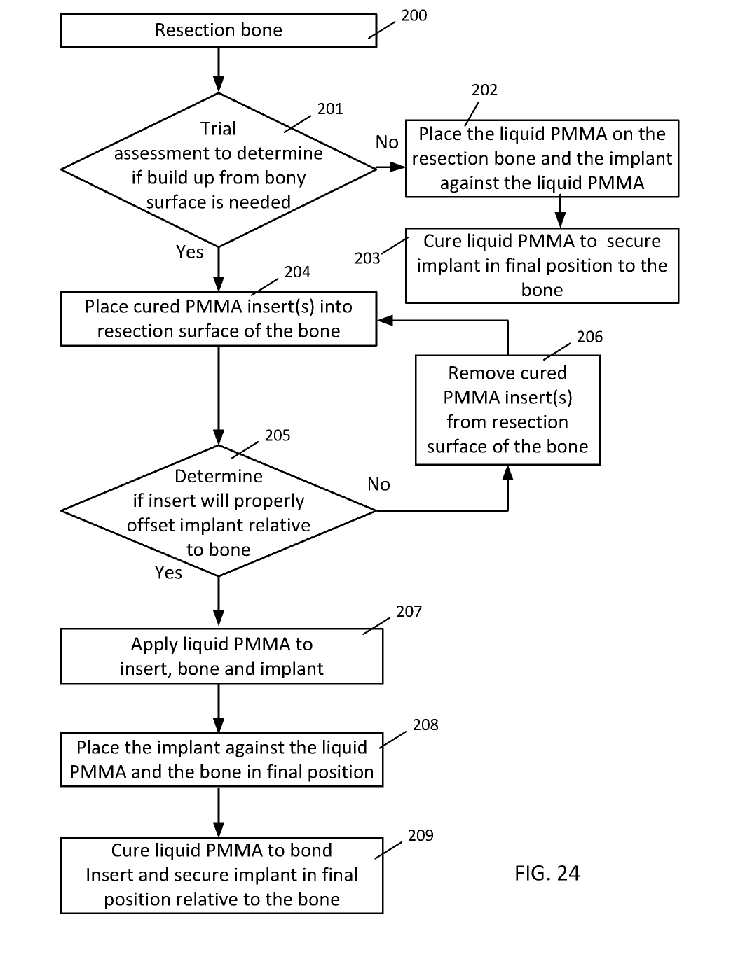

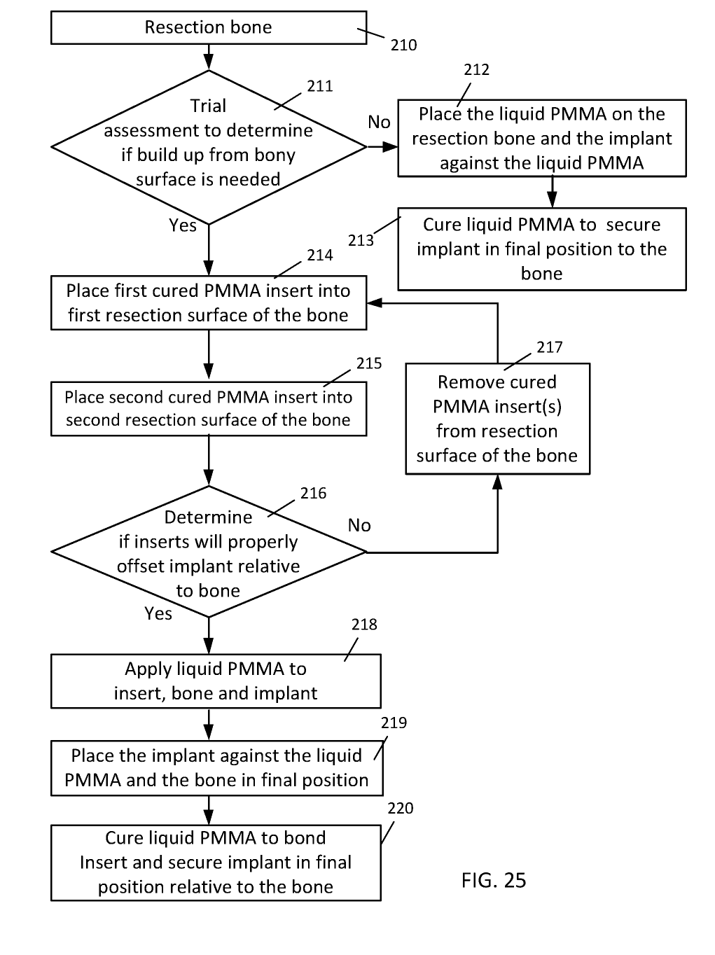

FIGS. 24-26 illustrate example flow charts describing the steps used to attach implants to a resectioned bone. With reference to the flowchart in FIG. 24, a bone is first resectioned 200. Trialing is then performed to determine if the implant will be properly positioned relative to the bone or if build up from the bony surface is needed 201. The trialing can be a test of the resection to determine if the position is correct. The trialing can depend upon the type of joint being repaired and can involve joint motion testing. The trialing will be described in more detail later. If the resection bone is proper and no build up from the bony surface is needed, liquid PMMA can be placed on the resection bone and the implant can be placed on the liquid PMMA and the bone 202. The liquid PMMA can then cure to secure the implant in the final position on the bone 203. If build up of the bony surface is needed, one or more cured PMMA inserts are placed into a resection surface of the bone 204. The surgeon can then determine if the one or more PMMA inserts will provide the proper offset 205. In some embodiments, a surgeon can use a tool such as a gauge to check the offset of the implant relative to the bone. In other embodiments, the implant can be placed against the inserts to determine the offset of the implant relative to the bone. The implant placed against the insert can be trialed for range of motion and stability to determine clinical adequacy of the correction of the implant relative to the bone. Alternatively, any other measuring method can be used to determine the offsets of the inserts. If an offset error is determined, the cured PMMA inserts can be removed from the resection surface of the bone 206 and cured PMMA inserts that provide different offsets are inserted into the resection surface of the bone 204.

In an embodiment, the surgeon can have a number of PMMA inserts that have different offset sizes. For example, the different PMMA inserts can be sized in 1 mm or other dimensional increments. In use, the user can insert the stem of the inserts and determine that the offset is the wrong length and then find a proper length offset insert based upon trial and error. In an embodiment, a surgeon can use a kit of paired inserts that can include various length offsets. In an embodiment, the inserts can be clearly marked so that the surgeon will know the different offsets of the different insert sizes which can improve the efficiency of the described procedures. The offsets of the inserts in a kit can range from 1 mm-15 mm in 1 mm increments or any other suitable range of distances and increments. So there can be 15 or more inserts each having a different offset distance. For example, the inserts can have dot markings that indicate the offset distance with each dot indicating an additional 1 mm offset. In other embodiments, the inserts can be numerically marked or color coded based upon the offset distance.

If the inserts provide the proper implant offset relative to the bone, liquid PMMA cement can be applied to the inserts, bone and implant 207. The implant is placed against the liquid PMMA which fills all gaps between the resection surface of the bone and the implant 208. In some embodiments, the liquid PMMA can be applied with a tool such as a brush or spatula to the contact surfaces of the stem sections with the insert and the implant. Liquid PMMA can also be injected with a tool such as a liquid PMMA injection gun through a nozzle into a gap between the resection surface of the bone and the implant to fill this space. Thus, the liquid PMMA can be applied to the inserts, bone and implant in various different ways. The liquid PMMA fills this space, cures and hardens to bond to the cured PMMA inserts on the first and second resection surfaces. The bonding of the liquid PMMA to the cured PMMA inserts create a high strength mechanical connection between the bone and the implant 209.

The use of cured PMMA inserts provides several benefits. The inserts provide a means for correcting resection errors when excess bone material has been removed. The physical strength of the PMMA connection to the bone is also improved because the cured PMMA inserts penetrate into the bone resulting in a stronger connection than that provided by liquid PMMA without the cured PMMA inserts. The chemical composition of the cured PMMA inserts and the liquid PMMA cement can be identical or substantially similar. When the liquid PMMA cures around the cured PMMA inserts, the solid structure formed is substantially homogeneous and the mechanical properties such as tensile, compression and shear strengths are uniform or nearly uniform across the cured liquid PMMA and PMMA insert regions. The radiopacity of the PMMA insert matches that of the liquid curing cement such that the radiographic appearance of the joint is not altered from standard technique. The PMMA composition of the insert does not interfere with techniques for implant removal during potential future revision surgery.

In joint arthroplasty, liquid PMMA cement rarely penetrates more than several millimeters into boney surfaces. The cured PMMA inserts can easily penetrate much further into the bone than liquid cement with the shaft of the insert acting as a solid column of PMMA. When liquid PMMA cement is applied to the PMMA insert, the resulting construct can create a greater strength mechanical bond between the bone and the bone implant than the mechanical bond of the bone to the bone implant with only PMMA cement without the PMMA inserts are used.

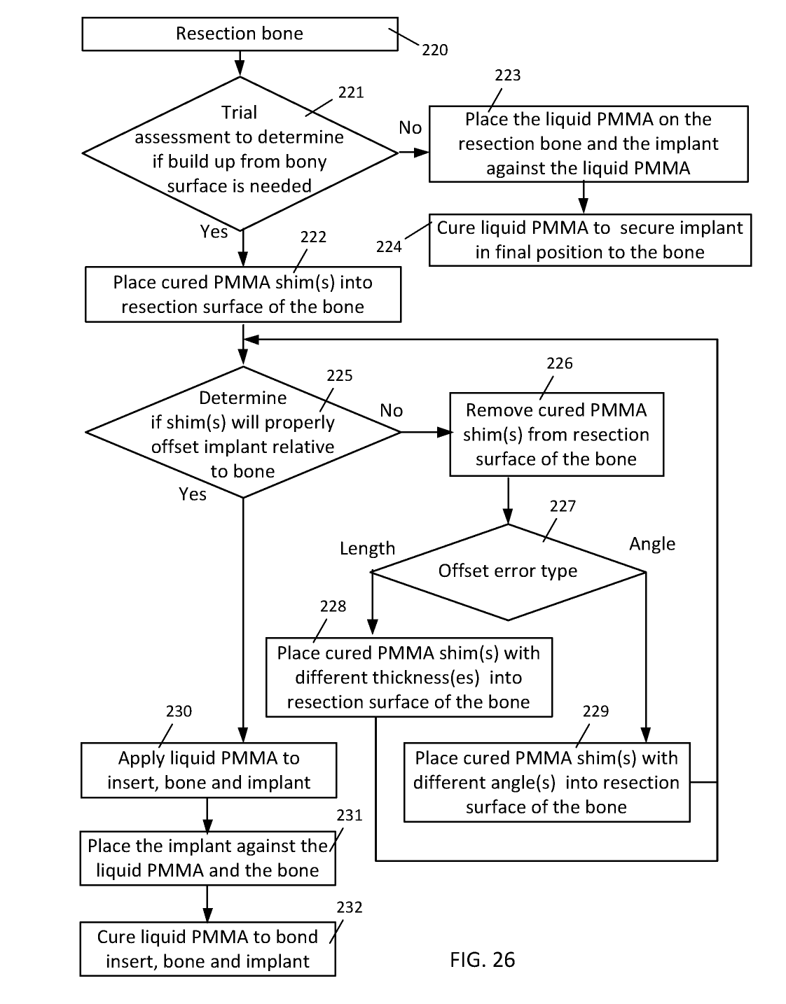

With reference to FIG. 26, a flowchart for coupling an implant to a bone with inserts on multiple resection surfaces. In this embodiment, the bone is resectioned forming multiple resection surfaces 210. Trialing is then performed to determine if the implant will be properly positioned relative to the bone or if build up from the bony resection surfaces is needed 211. If the resection bone surfaces are properly positioned and no build up from the bony surface is needed, liquid PMMA can be placed on the resection bone and the implant can be placed on the liquid PMMA and the bone 212. The liquid PMMA can then cure to secure the implant in the final position on the bone 213. In build up is needed to the resection surfaces, a first cured PMMA insert is placed in a first resection surface of the bone 214 and a second cured PMMA insert is placed in a second resection surface of the bone 215. The surgeon can then determine if the first and second inserts will properly position the implant offset relative to the bone 216. If the offset is incorrect, the inserts that need to be replaced are removed from the bone 217 and replacement first and/or second inserts are placed into the bone. If the inserts provide the proper implant offset relative to the bone, liquid PMMA cement can be applied to the inserts, bone and implant 218. The implant is placed against the liquid PMMA which can fill all gaps between the resection surface of the bone and the implant 219. The liquid PMMA cures and hardens to bond to the cured PMMA inserts on the first and second resection surfaces. This creates a high strength PMMA structure and secures the implant to the bone 220.

As discussed above, the inventive method can be used to make length and angular adjustments to bones, such as knee implants bonded to femur bones. FIG. 26 illustrates a flow chart describing a process for making length and/or angular corrections to the implant offset relative to the bone. The bone is resectioned 220 as described. In an embodiment, the resection surfaces can be an MFC resection surface and an LFC resection surface. Trialing is then performed to determine if the implant will be properly positioned relative to the bone or if build up from the bony resection surfaces is needed 221. If the resection bone surfaces are properly positioned and no build up from the bony surface is needed, liquid PMMA can be placed on the resection bone and the implant can be placed on the liquid PMMA and the bone 223. The cured PMMA inserts are then placed into the resection surfaces of the bone 224.

If build up is needed on the resection surfaces, the surgeon can insert implants and trial the implants to determine if the implant will properly offset the implant in length and angle relative to the bone 225. If there is an offset error, one or more of the cured PMMA inserts can be removed from the resection surface 226. The surgeon can determine if the error is in length and/or angle 227. If the implant offset length is incorrect, cured PMMA inserts can be placed in the resection surfaces of the bone to correct the offset length 228. The change in offset can be controlled by using different thickness MFC and LFC inserts that can have the same offset change, i.e. both MFC and LFC inserts can be shorter or longer than the original inserts to maintain the same offset angle. If the offset angle is in error, replacement inserts having different thicknesses can be inserted into the resection surfaces of the bone 229. Once the inserts that provide the proper angular and length offset are found, liquid PMMA can be applied to the insert, bone and implant 230. The implant can be placed against the liquid PMMA and the bone 231. The liquid PMMA can also fill the voids or gaps between the inserts, bone and implant. The liquid PMMA can cure to bond to the cured PMMA inserts and form a high strength structure to secure the implant to the bone 232.

As discussed, the inserts can have various different configurations. For example, in different embodiments the inserts can be tacks, rods, shims and/or any other suitable insert structure. Each of these insert configurations can have different component features and details of some possible implants will be described below.

### TACK INSERTS

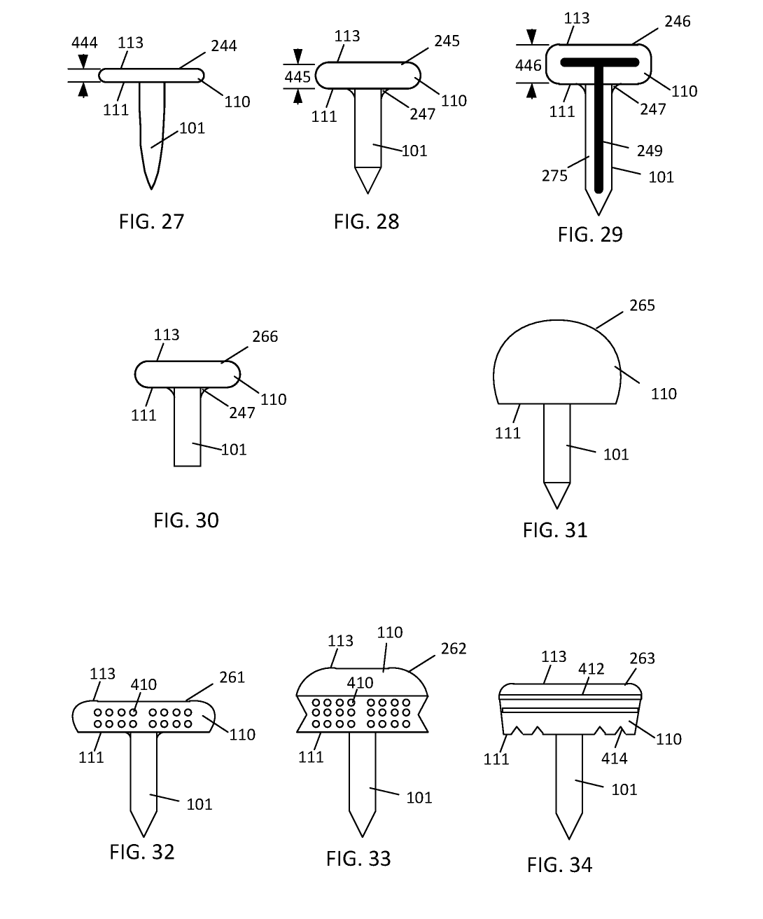

The tack can have a general geometry of a cap, a stem that is coupled to the cap and a stem taper section at the distal end. Examples of different cured PMMA tack insert embodiments are illustrated in FIGS. 27-42. The illustrated cured PMMA tack inserts have a stem 101 and/or a cap 110 that can include a Poly(methyl methacrylate) (PMMA) pre-cured cement outer surface that can penetrate the host bone and bond to the liquid PMMA cement.

With reference to FIG. 27, in a basic embodiment, a tack insert 244 can include a cap 110 and a stem 101 in direct physical contact with the cap 110. The stem 101 can be an elongated rod having an end and a tapered body that forms a sharp distal tip. A proximal end of the stem 101 is coupled to the cap 110. The center axis of the stem 101 can be perpendicular to a plane defined by the first surface 111 and/or second surface 113 of the cap 110. The stem 101 can have various different shapes including a rounded tapered tip, a sharp tip and a sharp tip that can gradually increase in cross section size to a wider diameter at the neck portion. The stem 101 can have a thin cross section that is inserted into the bone and the cap 110 can function as a mechanical stop so that when, the stem 101 is pressed into the bone and the first surface 111 of the cap 110 contacts the external surface of the bone. As discussed, an implant can be placed against the second surface 113 of the cap 110. Thus, the offset distance of the tack insert 244 can be defined by the distance 444 between the first surface 111 and the second surface 113 of the cap 110.

With reference to FIG. 28, another tack insert 245 is illustrated which has a thicker cap 110 having a longer distance 445 between the first surface 111 and the second surface 113 and FIG. 29 illustrates a tack insert 246 having a still longer distance 446 between the first surface 111 and the second surface 113 of the cap 110. Thus, the tack insert 246 in FIG. 28 provides a longer length implant offset than the tack insert 245 in FIG. 27 which provides a longer length implant offset than tack insert 244 in FIG. 27. In an embodiment, a plurality of tacks can be available during a bone surgery to provide the required offset. For example, the tack inserts can have different cap thicknesses in 1 mm increments. Thus, during a surgical procedure, the surgeon can perform trialing using different tack inserts so that the proper tack thickness can be determined. If the tack inserts are available in 1 mm increments, the implant will be able to be positioned within 1 mm of the correct implant offset distance from the bone. The trialing process will be described later in this application.

The shapes of the stems 101 can include a tapered section which creates a compression zone when inserted into the bone and a uniform cross section zone. In FIG. 27, the stem 101 can be tapered along the entire length which compress against the bone during the insertion of the stem 101. With reference to FIGS. 27 and 28, the stem 101 can include a conical section at the distal end that forms a sharp tip and a uniform cylindrical section along the middle and proximal portions of the stem 101. During the insertion process, the tapered sections of the stem 101 can press outward against the inner diameter of the hole in the bone compressing the bone outward as the cross section diameter of the tapered section enters the bone. This compression of the bone by the stem 101 can create a seal that can resist the movement of fluids such as liquid PMMA from flowing through this seal contact area. Once the tapered section has been pressed into the bone, the cylindrical section will not further compress against the bone during the insertion.

In some embodiments, it can be important for the tack to seal the PMMA liquid within the hole formed in the bone by the tack insert. With reference to FIG. 28, the diameter of the stem 101 connection adjacent to the cap 110 of the tack insert 245 can be wider than the outer diameter of the rest of the stem 101. More specifically, the junction between the stem 101 and the cap 110 can also include a radial expansion 247 which effectively expands the cross section of the stem 101 at the junction with the cap 110. The radial expansion 247 can be straight chamfer or a curved fillet between the stem 101 and the cap 110. When the tack insert 245 is inserted into the bone, the wider cross section will expand outward at the radial expansion 247 to create a physical seal with the bone. When the tack insert 245 is fully inserted into the bone hole, the wider diameter at the radial expansion 247 can have a tight fit with the upper edge of the hole when the tack is fully inserted. This tight fit seal can prevent liquid PMMA cement in the bone from escaping which can allow all of the internal liquid PMMA to cure and form a proper bond between the tack insert 245 and the bone.

As discussed, the inserts include a cured Poly(methyl methacrylate) (PMMA) outer surface. The cured PMMA material can penetrate and bond to the host bone and also bond to the liquid PMMA cement. The stem 101 and/or cap 110 can be made of a homogeneous cured PMMA material. However, in other embodiments, the inserts can be a composite construction that includes cured PMMA in combination with a different substrate material(s).

With reference to FIG. 29, the tack insert 246 can include a tack shaped substrate 249 which can be any non-PMMA material. For the example, the substrate 249 can be a metal such as stainless steel, titanium or any other suitable metal material. Alternatively, the substrate 249 can be made of a non-PMMA polymer material. The composite material implants can be useful when higher strength tack inserts 246 are necessary. For example, the shear strength of the PMMA insert can be improved by using a metal or polymer substrate. The substrate 249 is at least partially surrounded by cured PMMA 275. This construction can be achieved in various ways. For example, the tack implant 246 can be fabricated by placing the substrate 249 in a mold and that is then filled with liquid PMMA cement which is cured to form the cured PMMA insert 246. In other embodiments, the substrates 249 can be coated with the liquid PMMA which can cure to form the cured PMMA 275 around the substrates 249.

As discussed, the tack inserts can be inserted into the bone and the surgeon can perform trialing of the inserts using a trial implant to determine if the implant will be positioned with the correct offset and angle from the bone surface. If corrections need to be made, the surgeon must remove the tack implant(s) from the bone. In order to allow easier removal, the caps 110 can have over hang portions around the outer diameters which allow the surgeon to grasp the caps 110 and pull the tack inserts out from the bone. In FIGS. 27-29, the overhang features of the caps 110 are between the outer diameters and the first surfaces 111 of the caps 110. The surgeon can manually grasp these surfaces to pull the tack implant out of the bone. Alternatively, the surgeon may use a tool that can be placed between the bone and the over hang portions of the cap 110 to apply a removal force to the tack inserts and pull the tack inserts from the bone.

In different embodiments, the shape, thickness and geometry of the caps of the tack inserts can have any suitable geometry. For example, the caps can have an oval, circular, rectangular, triangular or any other cross section shape. The cross section can also be variable from the top to the bottom surfaces. For example, with reference to FIG. 30, an embodiment of a tack insert 266 can include a stem 101 can have a blunt distal end. With reference to FIG. 31, a tack insert 265 is illustrated having cap 110 with a spherical outer shape and a planar first surface 111 adjacent to the stem 101. The stem 101 can be inserted into bone and the first surface 111 can function as a stop against the outer surface of the bone. The spherical surface can provide a point of contact offset from the bone at any angle relative to the tack insert 265.

In other embodiments, the outer surface of the caps or heads can have relief or flow channels for excess PMMA liquids. The caps may also include, recesses, slots, grooves, holes and other features that can improve the binding with the surrounding or adjacent liquid PMMA cement. The cap can include a plurality of holes that extend from one side to an opposite side of the cap. The cap can have a plurality of slots in the side surfaces of the cap. Alternatively, the cap can have grooves or recesses formed in the lower and side surfaces. In all of these embodiments, the surrounding or adjacent liquid cement can flow into these cap surface features and harden. These features increase the surface area and provide additional structures that can capture and prevent the hardened PMMA from separating. The caps may also include holes that extend through the cap or recesses that the excess liquid PMMA cement can flow into and harden so that it does not flow out of the contact areas of the tack insert with the bone.

The bonding of the tack inserts to the bone and implant can be improved with greater interdigitation between the liquid PMMA cement, the tack inserts, the implant and the bone. The cap geometries can be designed to maximize cement penetration and increase the contact area between the cured PMMA insert and the liquid PMMA cement for improved interdigitation. In the tack insert embodiments illustrated in FIGS. 32-34, the surrounding or adjacent liquid cement can flow into these cap surface features and harden. These features increase the surface area and provide additional structures that can capture and prevent the hardened PMMA from separating. The caps may also include holes that extend through the cap or recesses that the excess PMMA liquid can flow into and harden so that it does not flow out of the tack area.

With reference to FIG. 32, a tack insert 261 can include a cap 110 having a plurality of holes 410 that extend from one side to an opposite side of the cap 110 or only partially into the cap 110. With reference to FIG. 33, a tack insert 262 can include a cap 110 having a more complex geometry. The cap 110 can which can have an “hour glass” shape which increases the contact surface area and also improves liquid PMMA cement penetration and bonding strength. The cap 110 can also include a plurality of holes 410 that extend from one side to an opposite side of the cap 110 or only partially into the cap 110. The outer diameter of the cap 110 can include a concave surface which can allow a surgeon to grasp and remove the tack insert 262. The cap 110 can be thicker to provide a greater offset of the implant relative to the bone. With reference to FIG. 34, in another illustrated example, a tack insert 263 can include a cap 110 can have a plurality of slots in the side surfaces of the cap. The cap 110 can also have recesses 414 such as grooves or other features formed in the first surface 111 of the cap 110. The outer diameter of the cap 110 can create an over hang that can allow the surgeon to grasp and remove the tack insert 263. As discussed, a PMMA liquid can be inserted into the bone hole before the tack insert 263 is placed in the hole. The PMMA liquid can surround the stem 101 and tip of the tack and secure these surfaces to the inner diameter of the bone. However, if there is excess PMMA liquid this material can only escape out of the top of the hole. In an illustrated embodiment, a first surface 111 of the cap 110 can have recesses 414 which can allow the PMMA liquid to flow into the recesses 414 which can prevent the PMMA liquid from escaping the cap contact area with the bone.

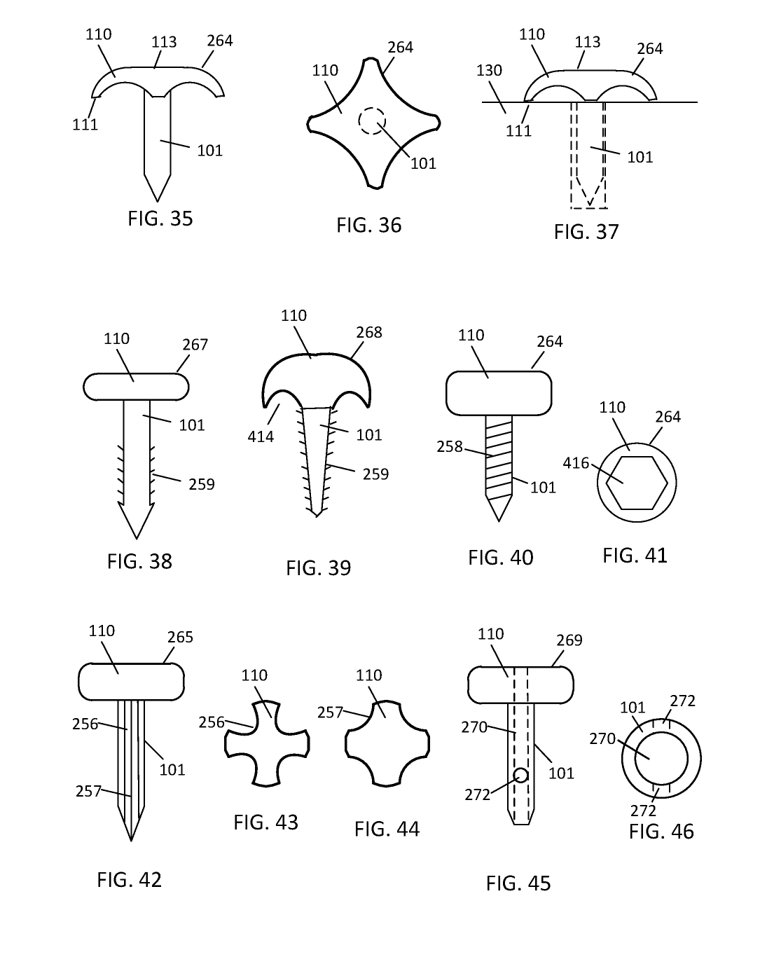

With reference to FIGS. 35-37, another embodiment of a tack insert 264 is illustrated. In this embodiment, the cap 110 can have a concave first surface 111 and a convex 113 upper surface. FIG. 35 illustrates a side view with the stem 101 extending away from the first surface 111 of the cap 110. The cap 110 may only contact the bone at several outer bone contact points of the cap 110. FIG. 36 illustrates a top view of the tack insert 264 which more clearly shows the shape of the cap 110 which can have several outer points and concave regions between the outer points. In this embodiment the cap 110 is not circular. With reference to FIG. 37, the tack insert 264 is illustrated after the stem 101 has been placed in a bone 130 with the cap 110 only contacting the bone 130 at a few points. In this embodiment, the liquid PMMA can flow between the cap 110 and the bone 130 to improve the bonding of the tack insert 264 to the bone 130.

In other embodiments, the tack inserts can be designed to provide various other features which may be useful for different types of surgical procedures. With reference to FIGS. 38 and 39, the stems 101 of the tack inserts can be designed to prevent removal from the bone once inserted. With reference to FIG. 38, the tack insert 267 can have a stem 101 with angled barbed protrusions 259 which are angled away from the tip of the stem 101. The angled protrusions 259 can increase the contact area with the cured PMMA and the liquid PMMA cement. When the stem 101 of the insert tack 267 is inserted into the bone, the angled protrusions 259 on the stem 101 and the tip of the stem 101 can compress against the stem 101 during insertion by the inner surfaces of the bone hole. However, when the stem 101 is pulled in a direction out of the bone, the outer ends of the angled protrusions 259 will contact the inner diameter of the bone holes and resist removal of the tack insert 267 from the bone.

With reference to FIG. 39, another embodiment of a tack insert 268 is illustrated with a tapered stem 101 that has angled protrusions 259 which will allow insertion but resist removal from the bone. The cap 110 can include lower recesses 414 such as grooves or other features formed in the cap 110 which can improve interdigitation with liquid PMMA cement. In the embodiments illustrated in FIGS. 38 and 39, the surgeon may determine the proper offset with other tack inserts and once the proper insert offset is known, the anti removal tack insert having the proper offset can be placed in the bone.

In different embodiments, the inventive tack can include a self-tapping screw configuration. With reference to FIG. 40, the stem 101 of the tack insert 264 can have helical threads 258. In this embodiment, the tack insert 264 can be rotated about a center axis of the stem 101 so the threads engage the inner diameter of a drilled bone hole or alternatively, the tack insert 264 can create the hole in the bone. The tack insert 264 can be driven into the bone by rotating the helical threads 258 until the cap 110 contacts the outer surface of the bone. The cap 110 of the tack insert 264 can have surface features that allow tools to engage the cap 110 so that a torque can be applied to rotate the stem 101. With reference to FIG. 41, a top view of a cap 110 of the tack insert 264 is illustrated which includes a hexagonal cross section recess 416 which can correspond to a hex wrench. The hex wrench can be inserted into the hex recess 416 and a torque can be applied to the wrench to rotate the tack insert 264. The threads 258 can engage the inner surface of the hole in the bone and drive the track insert 264 into the bone. The wrench can continue to rotate the tack insert 264 until the cap 110 contacts the outer surface of the bone. The surgeon can trial the offset of the tack insert 264 and if an adjustment needs to be made, the tack insert 264 can be removed by rotating the cap 110 in the opposite direction and the tack insert 264 can be replaced with a different tack insert having a different offset.

The stem of the inserts can have various features that can improve the interdigitation of the stem with the bone with the liquid PMMA cement. With reference to FIG. 42, in an embodiment the tack insert 265 can include a stem 101 having grooves 257 which extend along the length of the stem 101. The grooves 257 can allow liquid PMMA cement to flow in or out of the hole in the bone. As discussed, before a tack is inserted into the bone, the bone hole can be partially filled with liquid PMMA. The insertion of the tack can cause the PMMA liquid to be compressed and if there is insufficient volume between the tack and the hole, the PMMA liquid will be forced out of the hole. In the illustrated examples, the PMMA liquid can flow through the flow grooves and out of the hole in the bone. The grooves can allow the liquid PMMA cement to flow into the entire length of the stem 101.

FIGS. 43 and 44 illustrate cross sections of stems 101 having grooves 257, 258 which can provide additional contact surface areas for the liquid PMMA to flow, which can improve the bonding of the inserts to the bone. FIG. 43 illustrates deep grooves 256 in the stem 101 and FIG. 44 illustrates shallow grooves 257 in the outer surface of the stem 101 around the circumference. In some embodiments, the depths of the groove can be variable along the length of the stem 101. For example, the proximal portion of the stem 101 can be deeper to promote PMMA liquid flow into the groove 256 while the groove 257 can be shallower in depth at the distal end of the stem 101. In FIGS. 43 and 44, four grooves are shown around the circumference of the stem 101. In other embodiments, any other number of grooves can be formed in the circumference of the stem.

With reference to FIG. 45 a side view of an embodiment of a tack insert 269 is illustrated. The tack insert 269 can have a hollow cannulated stem 101 with a center passage way 270 that can also pass through the cap 110. FIG. 46 illustrates an end view of the stem 101 showing the passageway 270. In an embodiment, the tack insert 269 can be inserted into a bone and liquid PMMA can be pumped through the passageway 270 to the distal end of the stem 101. In an embodiment, a plurality of holes 272 can extend between the outer surface of the stem 101 and the passageway 270. The passageway 270 can allow liquid PMMA to be inserted between the bone and the tack insert 269 after the tack has been inserted into the bone. Thus, the tack insert 269 can be trialed off without applying liquid PMMA. If the tack insert 269 passes the trial process, liquid PMMA can be injected through the passageway 270 without removing the tack insert 269 from the bone.

### PMMA Rebar

In other embodiments of the present invention, multiple cured PMMA rods can be inserted into the bone surface. Liquid PMMA cement can be applied to the bone and the inserts and the implant can be placed on the liquid PMMA cement. The PMMA cement can harden and cure to chemically bond to the PMMA rods and mechanically bond the implant to the bone. The rods can be inserted into the bone at variable different angles which can further improve the strength of the bone bonding connection interface. In different embodiments, the rods can include various geometric features such as: tapered, threaded, posts, anchor constructs, etc. This system can provide improved cement interdigitation. The bonding surface of the implant can include fenestrations, grooves, roughness, etc. that can provide additional bonding surfaces for the PMMA cement.

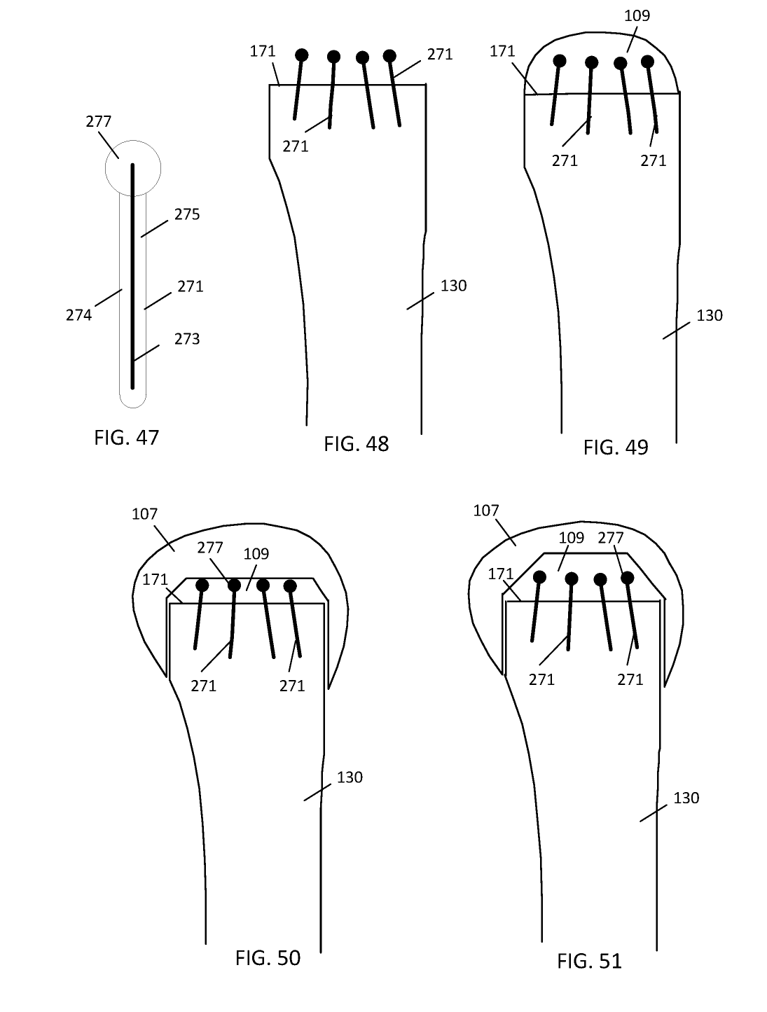

With reference to FIG. 47, an embodiment of a cured PMMA rod insert 271 can include a stem 274 and a head 277. In some embodiments, the cured PMMA rod inserts 271 can be composite structures for higher strength. Composite cured PMMA rod insert structures 271 can include a core substrate 273 made of materials stronger than cured PMMA, such as stainless, titanium, polymer, ceramic, metal, plastic etc. The core substrate 273 of the cured PMMA rod insert can be made of a material such as metal or high strength plastic which can have higher compression, tensile and shear strengths than a normal cured PMMA material. These core substrate 273 structures can then be covered with a PMMA material 275 which can completely or partially surround the core substrate 273. The PMMA 275 can be molded around the core substrate 273 structures or applied to the core substrate 273 structures as a cured PMMA coating 275.

The PMMA rod inserts 271 can come in various shapes and have various different structural features for example, the PMMA rod insert structures can include: a stem alone or possibly both a stem 274 and a head 277. In the illustrated example, the rod insert 271 can include elongated stems with a spherical shaped head 277 at one end of the stem 274. The head 277 can provide a larger surface area so that the stem 274 portion can be physically pushed into the hole in the bone or a bony surface so that the rebar PMMA rod inserts 271 function like a pushpin as previously described in the tack insert embodiments.

In other embodiments, the stem can be: solid, cannulated, fenestrated and the outer surface can be smooth, textured, threaded, grooved or have any other surface features. These surface features can provide improved grip to the cement and other adhesives. If the rebar device includes a head, the head can provide additional functional features. For example, if the stem portion is threaded, the head can have features that can allow a torque to be applied to the stem that can rotate the rebar device and drive the rebar device into the bone. For example, the upper surface of the head can have slots that can engage an end of a screw driver. Alternatively, the outer edges of the head can have surfaces that are parallel to the center rotational axis of the stem which can allow a wrench to apply a torque force to the head and stem.

With reference to FIG. 48, a plurality of rod inserts 271 are inserted into an exposed attachment surface 171 (surface not labeled) of the bone 130. The exposed surface 171 can be a resectioned surface as described above. However, in other embodiments, the exposed surface 171 of the bone can be an exposed bony surface which can be at least one of the following types of bone: a metaphyseal bone, a cancellous bone, a trabecular bone, a porous bone, or a sclerotic bone. The exposed surface 171 can be drilled or alternatively, the rod inserts 271 can be pressed directly into the exposed surface 171. The angles of the rod inserts 271 within the bone 130 can be variable so that the rod inserts 271 may not be parallel to each other. However, in other embodiments, the rod inserts 271 can be perpendicular to the exposed surface 171 and parallel to each other.

With reference to FIG. 49, a liquid PMMA 109 can be applied to the rod inserts 271, the exposed surface 171 and the holes formed in the bone 130. The liquid PMMA 109 can have sufficient viscosity to be manually pliable. It can be desirable to remove all air bubbles in the liquid PMMA 109 to maximize the strength of the PMMA when it cures. The implant 107 is placed on the liquid PMMA 109 which can fill the contact areas between the implant 107 and the bone 130. With reference to FIG. 50, the inner surfaces of the implant 107 may be in direct physical contact with the heads 277 of the cured PMMA rod inserts 271. With reference to FIG. 51, the implant 107 may not contact the cured PMMA rod inserts 271. The position and offset of the implant 107 relative to the bone 130 can be adjusted while the PMMA 109 is in liquid form. Because the implant 107 does not come into physical contact with the rod inserts 271, the implant 107 can be manually positioned. Once properly positioned, the implant 107 can be held in a stationary position relative to the bone 130 until the PMMA cures to bond the implant 107 to the bone 130 and inserts 271.

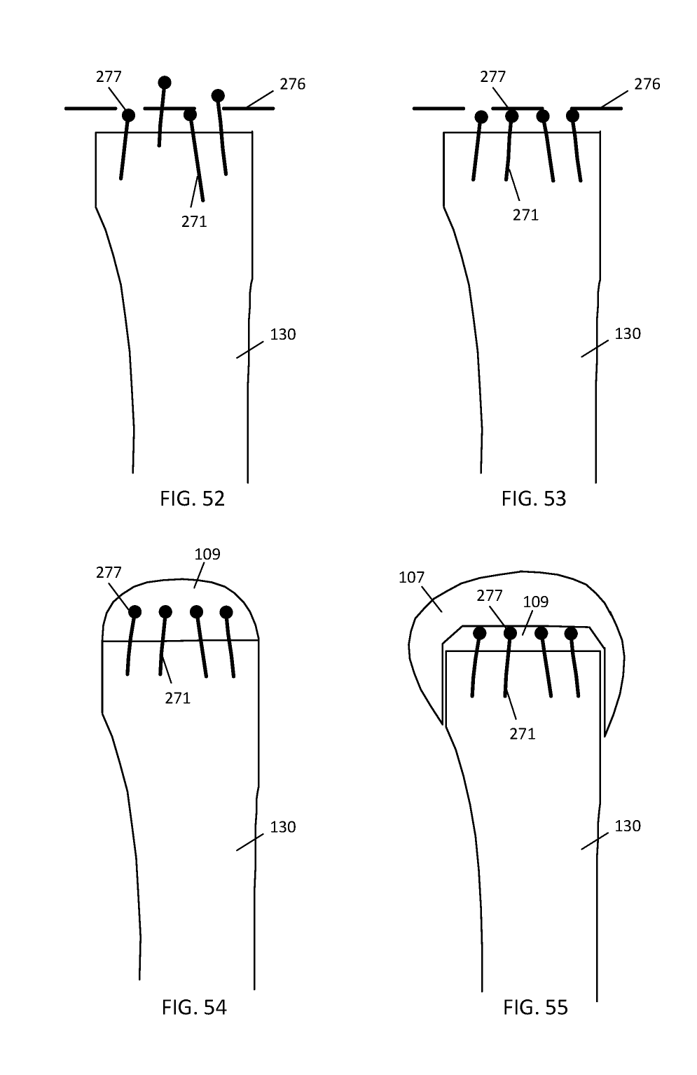

In other embodiments, the rod inserts 271 can be adjusted to control the offset of the implant relative to the bone. With reference to FIG. 52, the bone 130 is illustrated with a plurality of rod inserts 271 and a required offset plane line 276 is illustrated. Some of the rod inserts 271 extend beyond the offset plane line 276 while other rod inserts 271 can be below the offset plane line 276. With reference to FIG. 53, the rod inserts 271 that do not match the offset plant line 276 have been replaced with rod inserts 271 that match the offset plane line 276. Alternatively, the rod inserts 271 can be pressed farther into the bone to match the offset plane line 276. In the illustrated embodiment, the rod inserts 271 have heads 277 which can be aligned with the offset line 276 and contact the implant 107. With reference to FIG. 54, once the inserts 271 are properly positioned relative to the offset plane line 276, liquid PMMA cement 109 is placed on the rod inserts 271 and the exposed attachment surface 171 of the bone 130. With reference to FIG. 55, the implant 107 is placed on the bone 130 with the implant 107 in direct physical contact with the heads 477 of the rod inserts 271.

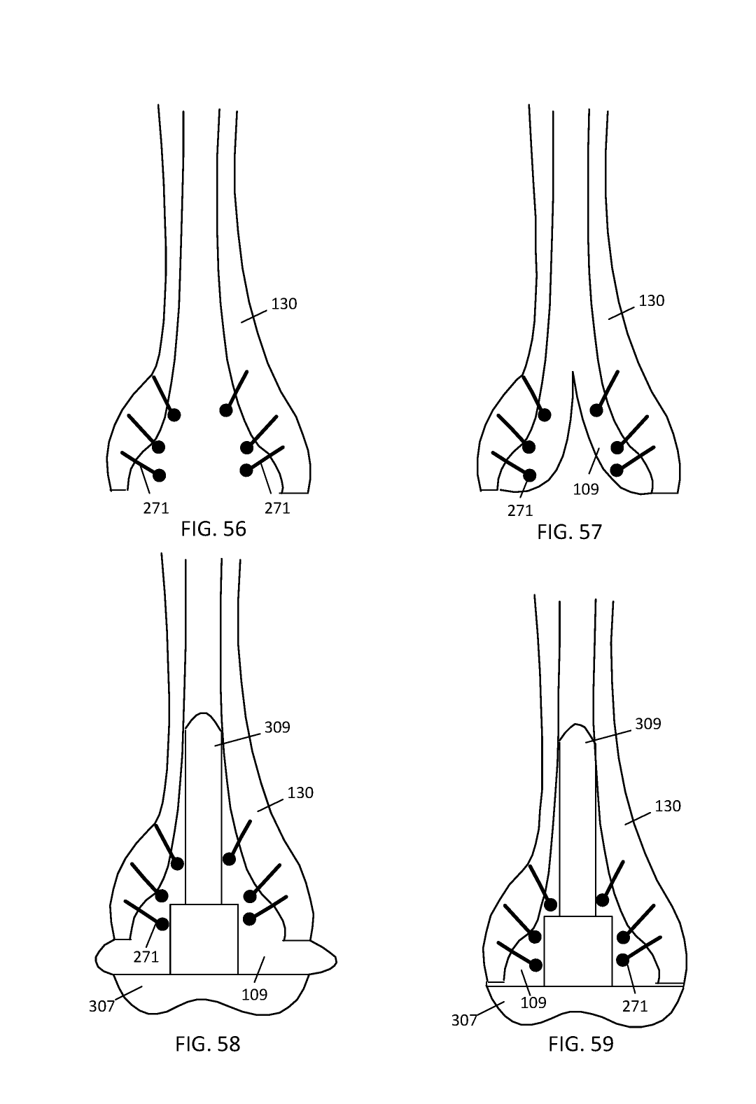

In other embodiments, rod inserts 271 can be used to improve the bonding of an implant 307 that extends into the bone 130. In this embodiment, the implant 307 may include an elongated portion 309 that is inserted into the bone 130 and is partially surrounded by the bone 130. With reference to FIG. 56, a bone 130 cross section is illustrated. An end of the bone has been resected and a plurality of rod inserts 271 placed on inner surfaces of the bone 130. In an embodiment, the rod insert 271 positions can be checked or trialed to determine if they are properly positioned within the bone 130. If any changes are required, the rod inserts 271 can replaced or adjusted. With reference to FIG. 57, once the rod inserts 271 are correctly positioned, liquid PMMA 109 can be applied to the inner surfaces of the bone 130 and the rod inserts 271. With reference to FIG. 58, the elongated portion 309 of the implant 307 is inserted into the bone 130 between the rod inserts 271 which can guide the elongated portion 309 into the bone 130. With reference to FIG. 59, the implant 307 has been fully inserted into the bone 130 and the liquid PMMA 109 can cure to bond the implant 307 to the bone 130 and rod inserts 271.

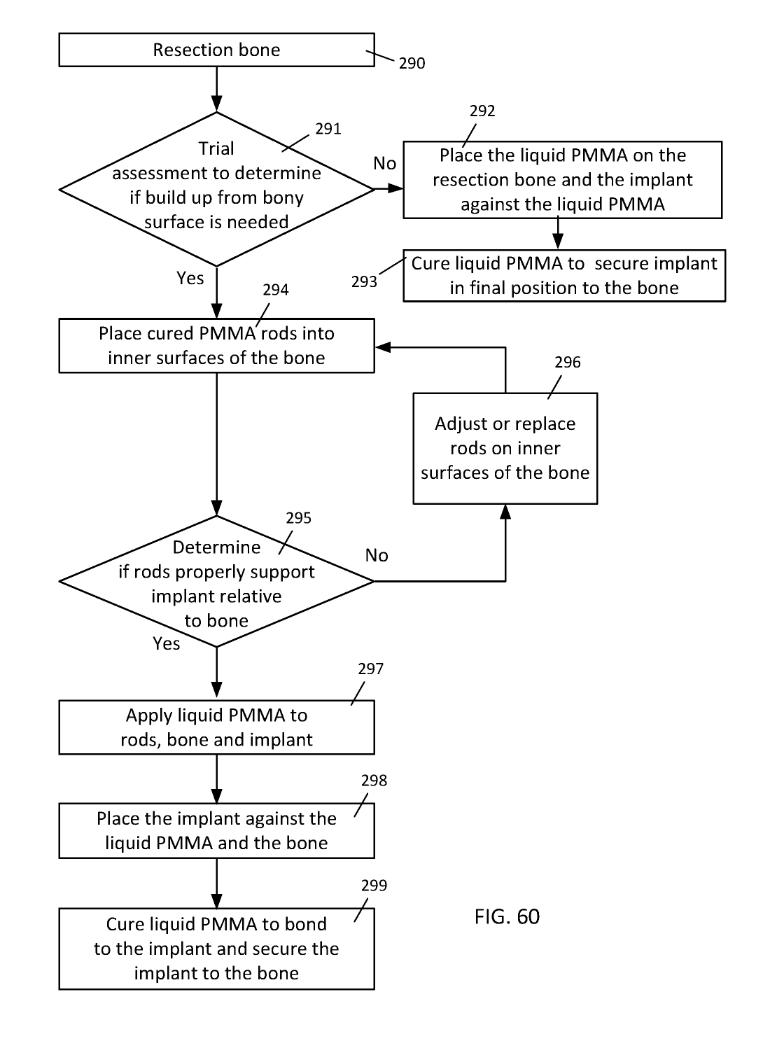

With reference to FIG. 60, a flow chart is illustrated describing the process steps for bonding implants to the bone using the rod inserts. A bone can be resectioned 290. Trialing is then performed to determine if the implant will be properly positioned relative to the bone or if build up from the bony surface is needed 291. If the resection bone is proper and no build up from the bony surface is needed, liquid PMMA can be placed on the resection bone and the implant can be placed on the liquid PMMA and the bone 292. If build up from the bony surface is required, the surgeon can then place cured PMMA rod inserts into the inner surfaces of the bone 294. The cured PMMA rod inserts can be trialed to determine if the rod inserts are properly positioned to support the implant offset relative to the bone 295. If the rod implants are not properly positioned, the surgeon can adjust or replace the rod inserts on the inner surfaces of the bone 296. Once the rod inserts are properly positioned, liquid PMMA cement can be applied to the rod inserts and bone 297. The surgeon can then place the implant against the liquid PMMA and the bone 298. The liquid PMMA cement can be cured to bond to the PMMA rod inserts and the implant can be secured to the bone 299.

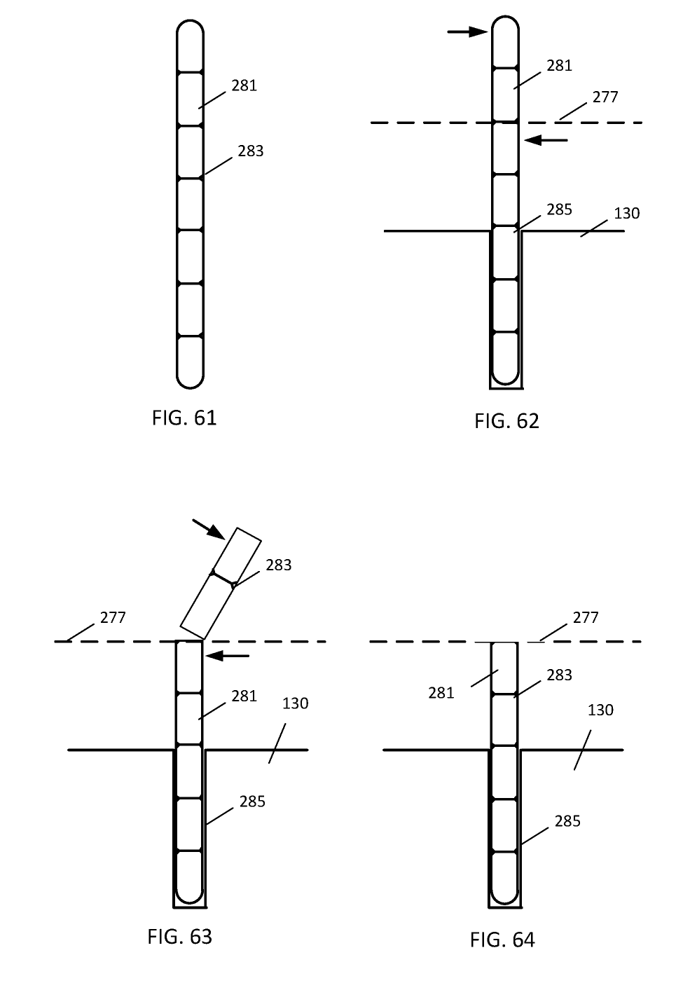

While some of the rod inserts have been illustrated as elongated rods with heads, in other embodiments, the rod inserts may be elongated stems without heads. For example, with reference to FIG. 61, in an embodiment the cured PMMA rod insert 281 can be scored at fracture lines 283 so that the rod insert 281 is breakable to shorten the length. This can be helpful when a rod insert 281 needs to be shortened. The surgeon can shorten the rod insert 281 to the desired length rather than removing and replacing the rod insert. The fractures lines 283 can be set at uniform positions along at least a portion of the cured PMMA rod insert 281. With reference to FIG. 62, the cured PMMA rod insert 281 can be inserted into a hole 285 in the bone 130 and a portion of the PMMA rod insert 281 can extend out of the exposed surface of the bone 130 and across a required offset plane line 287. The surgeon can identify the fracture line 283 that most closely matches the required offset plane line 277. With reference to FIG. 63, the PMMA rod insert 281 can be broken at the fracture line 283 on the required offset plane line 277 by applying a force to the top of the PMMA rod insert 281 while the body of the PMMA rod insert 281 is supported below the required offset plane line 277. In other embodiments, the PMMA rod insert 281 can be cut with a tool to the required length. With reference to FIG. 64, the PMMA rod insert 281 now matches the required offset plane line 277. The PMMA liquid can be applied to the PMMA rod insert 281 and bone 130 prior to attaching the implant 107 to the bone 130 as described above.

### SHIM INSERTS

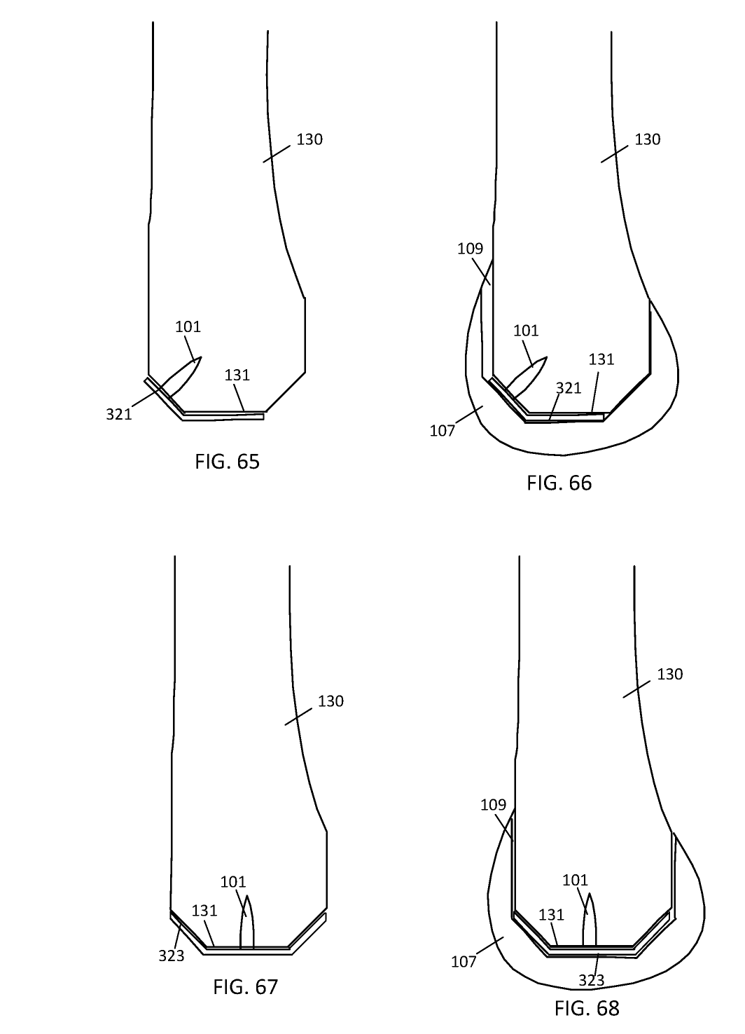

In other embodiments, the planar and/or angular cured PMMA shims can be used to adjust the implant offset relative to the bone. Shims can include a planar or an angled shim structure which may or may not be coupled to a stem that is inserted into the bone. The shims can be placed over one or more bony surfaces. With reference to FIG. 65, in an embodiment a cured PMMA shim 321 having a stem 101 that is pressed into an anterior resection surface 131 of a bone 130. The surgeon can do a trial off of the cured PMMA shim 321 using a trial implant (not shown). Based upon the trial results, the surgeon can determine if the PMMA shim 321 is the proper thickness or if the shim 321 needs to be replaced. If necessary, the shim 321 can be removed and replaced with a shim having a different thickness that provides a proper implant offset and angle. With reference to FIG. 66, once the proper size PMMA shim 321 is successfully trialed, liquid PMMA cement 109 can be applied to the implant 107, the cured PMMA shim 321 and the resection surfaces 131 of the bone 130. The liquid PMMA cement 109 can fill all of the spaces between the bone 130 and the implant 107 and cure to chemically bond to the cured PMMA shim 321 and mechanically bond the implant 107 to the bone 130.

With reference to FIG. 67 in other embodiments, a cured PMMA shim 323 can be designed to cover other bony surfaces 131 of the bone 130. In this example, the cured PMMA shim 323 can cover the anterior, distal and posterior bony surfaces 131 of the bone 130. The stem 101 in this embodiment is pressed into the distal bony surface 131. The surgeon can use a trial implant to perform trial testing on the PMMA shim 323 to determine if the shim 323 is the proper thickness to provide the proper implant offset or if the shim 323 needs to be replaced with a different sized PMMA shim. With reference to FIG. 68, once the proper PMMA shim 323 is found, the liquid PMMA cement 109 can be applied to the implant 107, cured PMMA shim 323 and (anterior, distal and posterior) resection surfaces 131 of the bone 130. The liquid PMMA cement 109 can fill all of the spaces between the bone 130 and the implant 107 and cure to chemically bond to the cured PMMA shim 323 and mechanically bond the implant 107 to the bone 130.

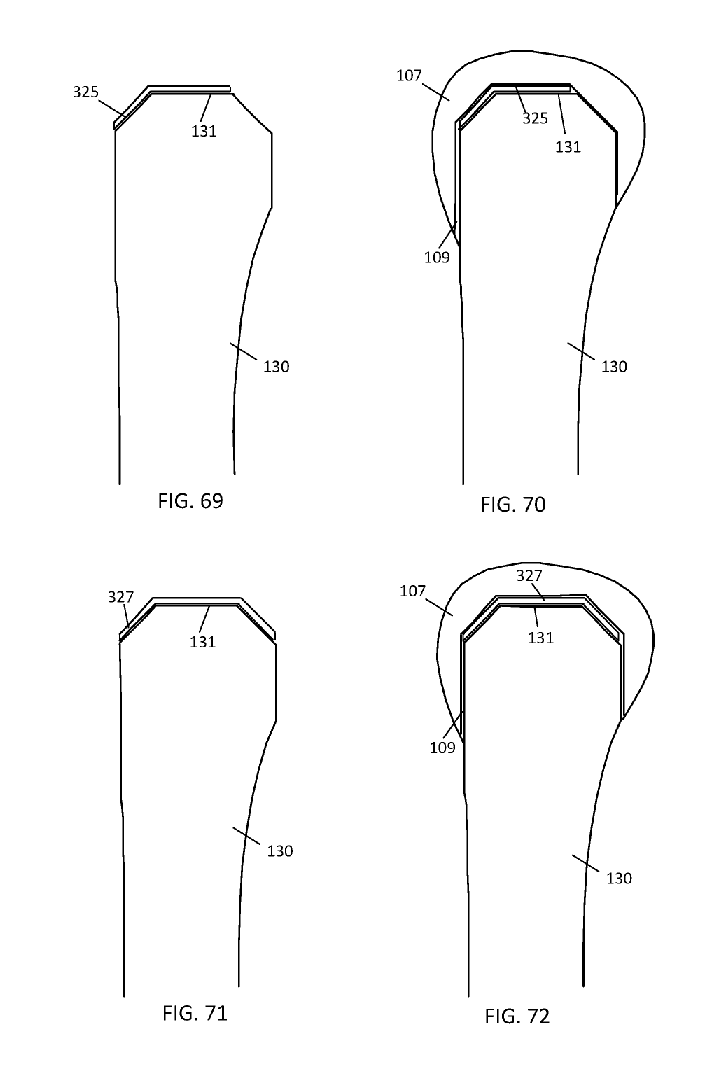

The cured PMMA inserts have been described as having stems which are inserted into the bone. However, in other embodiments, cured PMMA inserts can be placed on outer bony surfaces without any stem or other structures that are pressed into the bone. With reference to FIG. 69, a shim 325 that does not have a stem is placed on anterior and distal resection surfaces 131 of the bone 130. The surgeon can use a trial implant to perform trialing on the shim 325 and determine if the shim 325 provides the proper offset and angle for the implant or if the shim 325 needs to be replaced. With reference to FIG. 70, if the shim 325 passes the trialing requirements, liquid PMMA 109 can be applied to the resection surfaces 131, the insert 325 and the implant 107. The liquid PMMA 109 can cure and form a chemical bond with the PMMA insert 325 and mechanically bond the implant 107 to the bone 130.

FIG. 71 illustrates another embodiment of a cured PMMA shim 327 that covers anterior, distal and posterior resection surfaces of the bone and does not have a stem. The surgeon can trial off the shim 327 as described above and replace the shim 327 if necessary. Once the proper shim 327 is found, liquid PMMA 109 can be applied to the resection surfaces 131, the insert 325 and the implant 107. With reference to FIG. 72, the liquid PMMA 109 can cure and form a chemical bond with the PMMA insert 327 and mechanically bond the implant 107 to the bone 130.

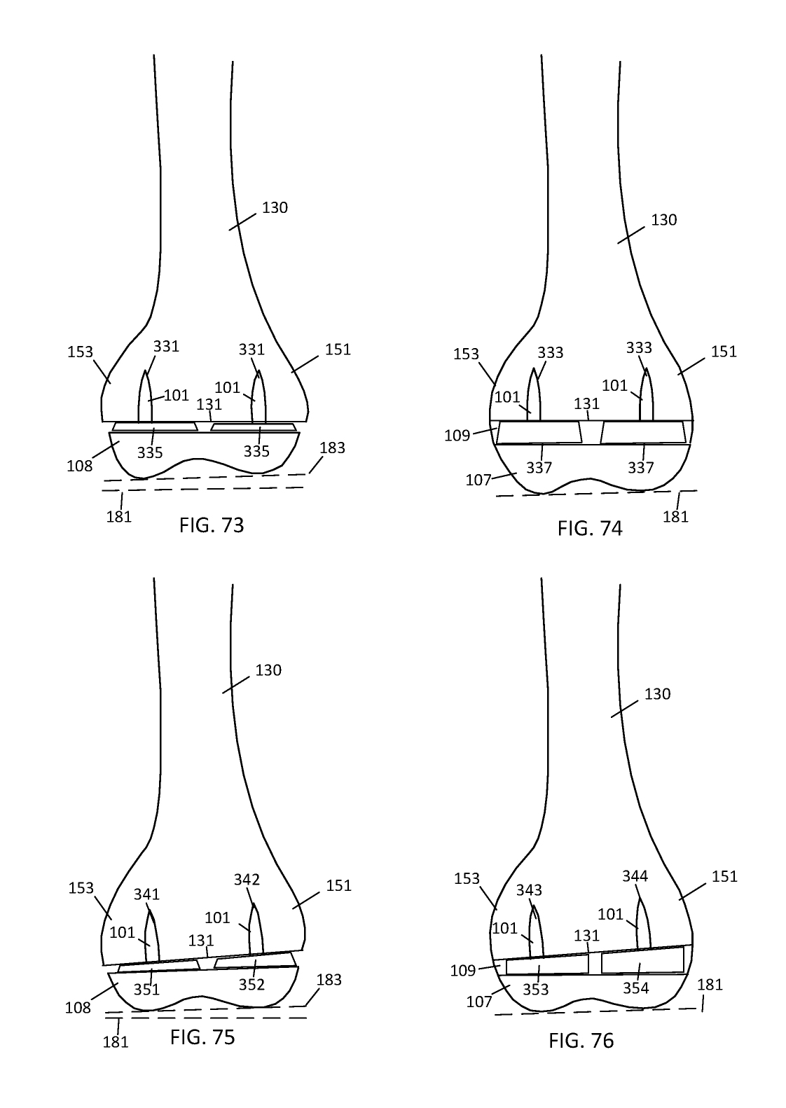

The shims can be used to correct length and angular offset of the implant relative to the bone. As discussed, the surgeon can test the offset of the inserts by performing trialing processing with a trial implant placed on the inserts. The trialing can include range of motion measurements for joints and tension testing. With reference to FIG. 73, the proper predetermined length offset of the implant relative to the bone 130 can be represented by line 181. However, in the illustrated embodiment, the offset line 183 provided by the implant shims 335 and the trial implant 108 is substantially shorter than the proper offset line 181. Based upon the trialing, the surgeon can determine that thicker implant shims are needed. With reference to FIG. 74, the length of the offset between the bone 130 and the implant 107 has been increased by removing the original shims (335) and replacing them with thicker shims 337 so the offset of the final implant 107 matches the proper predetermined length offset line 181. In other embodiments, if the offset position of the implant 107 needs to be shortened, the inserts can be replaced with thinner shims. In this embodiment, the angle of the resection surface 131 was correct, so the shims 337 can have the same thickness so that the angle of the implant 107 is not changed relative to the bone 130. Liquid PMMA 109 can be applied to the bone 130, shims 337 and implant 107 to chemically bond the PMMA cement 109 to the PMMA shims 337 and mechanically bond the implant 107 to the bone 130.

It can also be possible to correct angular offset errors of the resection bony surfaces. For example, with reference to FIG. 75, cured PMMA shims 351 and 352 are placed in the resection surface 131 of the bone. The shims 351 are trialed using a trial implant 108 and the surgeon may determine that the shims 351 result in a different offset angle 183 than the proper offset line 181. The offset angle of the implant 107 relative to the bone 130 can be corrected by replacing the shims 351 and 352 with PMMA shims 353 and 354 that have different thicknesses and angles. With reference to FIG. 76, the replacement shims 353 and 354 can correct the offset position of the final implant 107 to the angle and position that match the correct predetermined offset line 181. Alternatively, if the surgeon needs to angle the implant 107 more towards the medial side, the shim placed in the MFC 151 can be thinner than the shim in the LFC 153.

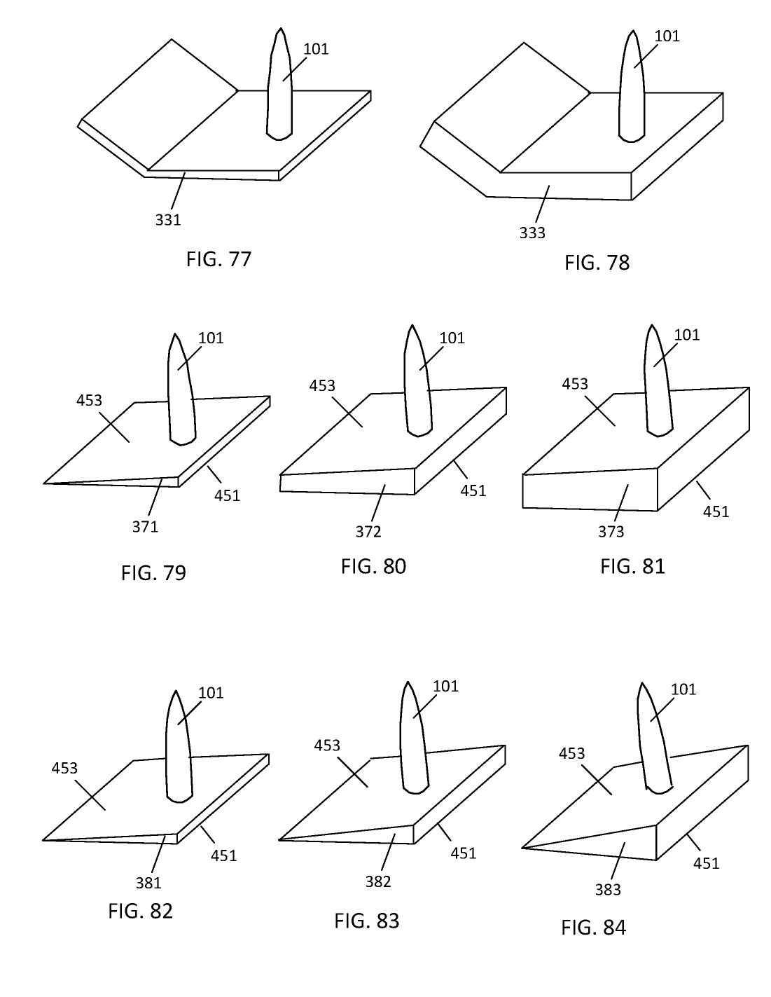

In an embodiment, when a bone is resected, jigs can be used to create specific cuts in the bone resulting in specific shapes and angles of resection bony surfaces. The resectioned bone can be trialed with a trial implant to determine if the resection surfaces are correct based upon range of motion, laxity and stability testing. If the bone is resectioned perfectly and the trial testing is successful, the shim implant can be mechanically bonded to the resectioned bone with liquid PMMA cement. However, if there are any errors in the resectioning and the bony surfaces needs to be built up to correctly offset the implant, shim inserts can be used to make these corrections. In an embodiment, a set of shims can be manufactured specifically to match the angles and shapes of the implant and bony surfaces. The shims can be made at various thicknesses and angles to allow a surgeon to make any necessary corrections to properly position the implant during surgery. FIGS. 77-84 illustrate examples of shims which can be used to make length and angle corrections to the bony surfaces where the implant will be mounted.

FIGS. 77 and 78 illustrate shims 331, 333 that can cover multiple resection surfaces. The stem 101 can be inserted into the bony surface and the shim can cover two adjacent resection surfaces. The surgeon may determine that the offset needs to be increased. If a little offset is needed, the thinner PMMA shim 331 illustrated in FIG. 77 can be used and if a longer offset is needed, the thicker PMMA shim 333 illustrated in FIG. 78 can be used. For example in an embodiment, the trial implant can be tested on the resectioned bone. The PMMA shim 331 can be attached by pressing the stem 101 into the bone. Different thickness shims can be trialed until the proper PMMA shim is found.

With reference to FIGS. 79-81, embodiments of angled cured PMMA shims 371, 372, 373 are illustrated. The shims 371, 372, 373 can each have the same relative angle between the first surface 451 and the second surface 453. However, the angled cured PMMA shims 371, 372, 373 have different thicknesses. As discussed, a trial implant can be tested on the resection surfaces of the bone and the surgeon can perform a trial assessment of the shim. If surgeon determines that shim inserts are needed, the surgeon can select one of the cured PMMA shims 371, 372, 373. The selected shim can be trialed and if the shim passes the trial assessment, liquid PMMA cement can be applied to the shim, bone and implant to chemically bond to the shim and mechanically bond the implant to the bone.

With reference to FIGS. 82-84, embodiments of angled cured PMMA shims 381, 382, 383 are illustrated. The shims 381, 382, 383 can each have a different angle between the first surface 451 and the second surface 453. A trial implant can be tested on the resection surfaces of the bone and the surgeon can perform a trial assessment of the shim. If surgeon determines that an angled PMMA shim insert is needed, the surgeon can select one of the cured PMMA shims 381, 382, 383. The selected shim can be trialed and if the shim passes the trial assessment, liquid PMMA cement can be applied to the shim, bone and implant to chemically bond to the shim and mechanically bond the implant to the bone.

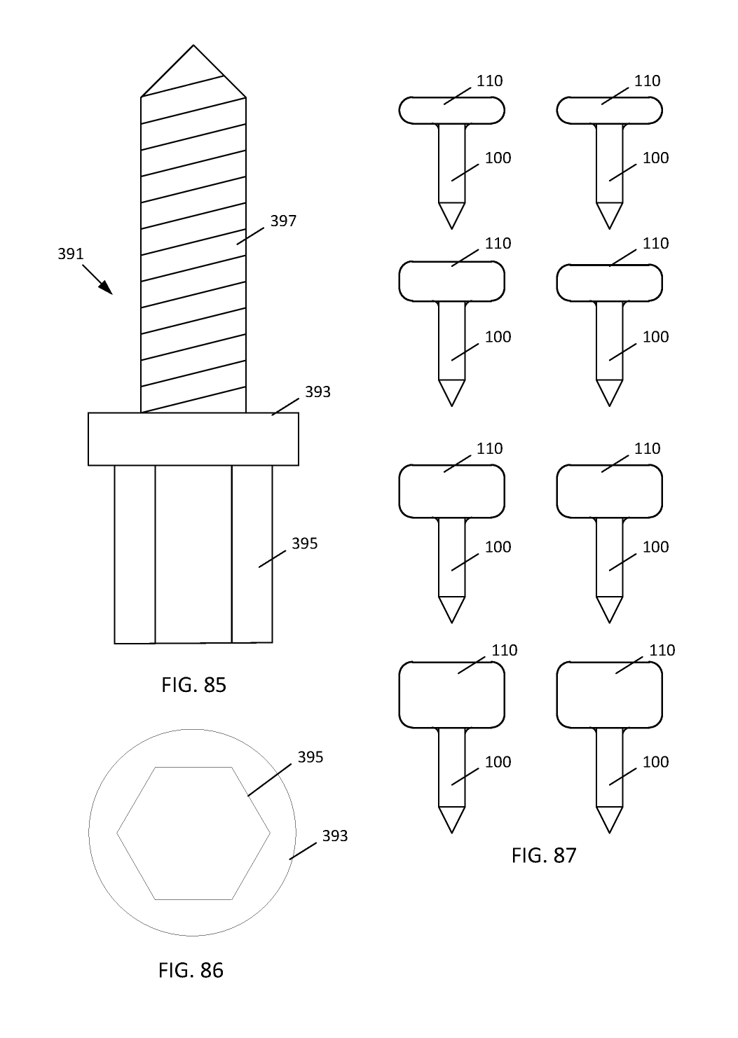

In different embodiments the described cured PMMA insert system can be provided to doctors in the form of a PMMA insert kit which can include any combination of components. The kit may also include a stepped drill bit which can be used to form holes for the elongated rod portions of the cured PMMA inserts. The drill bit can include a sharp cutting portion and a smooth step that has a larger diameter. When a bone is drilled, the sharp cutting portion will form the holes but the drill bit will stop removing bone material when the smooth step edge contacts the outer surface of the bone. The drill bit can produce uniform diameter and depth holes in bones. For example with reference to FIGS. 85 and 86, in an embodiment the stepped bone drill 391 which includes a helical cutting portion 397 and a stop step 393. A drive portion 395 of the bone drill 391 opposite the cutting portion 397 can have a hexagonal cross section which can be attached to a drill mechanism. With reference to FIG. 87, a PMMA insert kit can include a plurality of PMMA tack inserts 100 that can have many different head thicknesses. In an embodiment, the PMMA tack inserts 100 may have different thicknesses which vary by 1 mm increments, such as 1 mm, 2 mm, 3 mm, etc. The PMMA insert kit can provide 2 to 4 tack inserts per head thickness size. In this illustrated example, the heads of the tacks can be flat planar meaning that the planes defined by the upper and lower surfaces of the heads can be parallel.

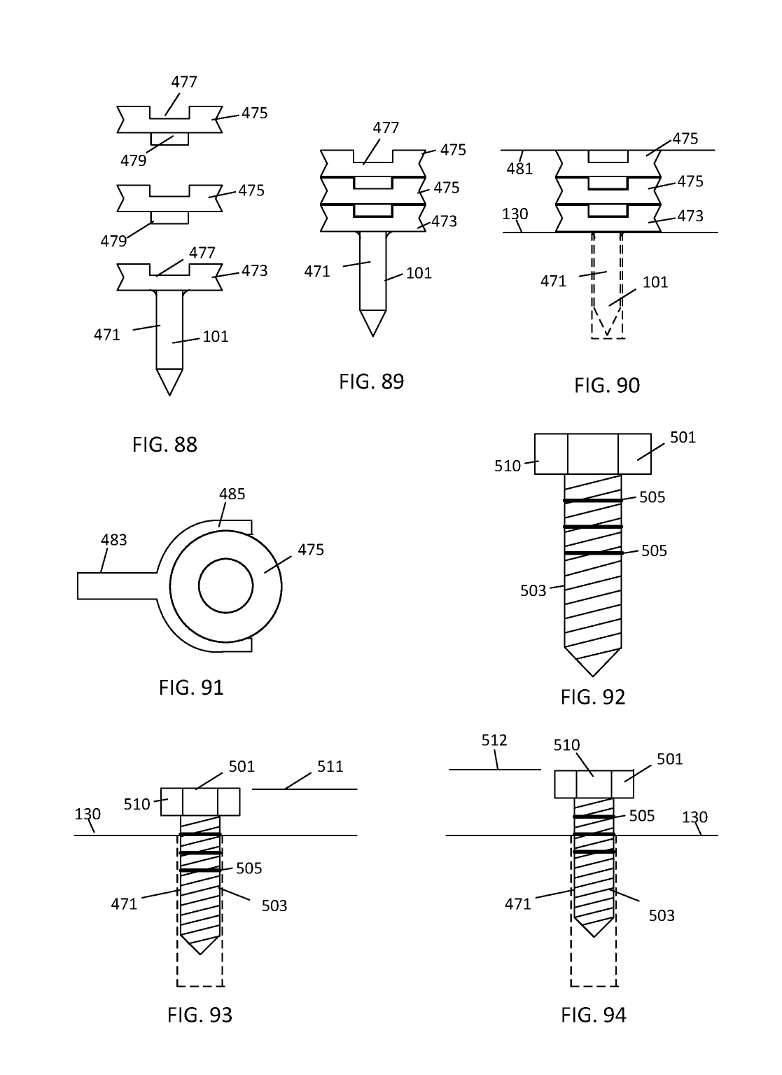

With reference to FIGS. 88-90, in other embodiments the tack inserts 471 can have a modular design which can allow the caps 473 of the cap inserts 471 to be adjusted in thickness by adding cap attachments 475. In an embodiment, the caps 473 and cap inserts 471 can include recesses 477 and the cap inserts 471 can include coupling features 479. If the cap 473 thickness of the tack insert 471 does not provide a sufficient offset, one or more cap inserts 471 can be attached to the cap 473. FIG. 88 illustrates the tack insert 471 and separated cap inserts 471. The coupling features 479 on the cap inserts 471 can be placed in the recesses 477 to increase the assembly cap offset. With reference to FIG. 89, two cap attachments 475 have been attached to the cap 473 of the tack insert 471 with the coupling features 479 inserted into the recesses 477. The cap 473 and the cap attachments 475 can include concave surfaces which can allow the surgeon to easily grasp the cap 473 and cap attachments 475 and adjust the offset of the cap 473 and cap attachments 475. With reference to FIG. 90, the tack insert 471 with cap attachments 475 is inserted into the bone 130 and a trial implant 481 is placed against the upper cap attachment 475 to perform trial assessment. If the tack insert 471 with cap attachments 475 provide the correct implant offset, liquid PMMA can be applied to the tack insert 471 and cap attachments 475 assembly and the bone and implant. The liquid PMMA can cure and chemically bond to the PMMA insert and create a mechanical bond between the bone implant and the bone.

In an embodiment with reference to FIG. 91, an insert kit can include a tool 483 having a fork mechanism 485 that can engage the concave surfaces on the outer diameters of the cap and the cap attachments 475. The tool 483 can be used to easily couple or separate the cap attachments 475 as necessary based upon the trialing assessment.

With reference to FIG. 92, an embodiment of a threaded cured PMMA insert 501 is illustrated. The PMMA insert 501 can have a cap 510 that can be rotated with a tool to drive the threaded stem into a bone. The threaded stem 503 can have visual markings which can allow the surgeon to know the offset of the PMMA insert 501. In this example, the visual markings 505 on the threaded stem are a plurality of lines which can be spaced at uniform distances. With reference to FIG. 93, the threaded PMMA insert 501 has been screwed into a bone to a depth that matches a first offset line 511. The surgeon can place a trial implant against the upper surface of the cap and perform trialing. If there is an error, the surgeon can make adjustments to the offset of the PMMA insert 501 by rotating in one direction to drive the threaded PMMA insert 501 further into the bone or in the opposite direction to move the PMMA insert 501 further out of the bone. With reference to FIG. 94, the surgeon may rotate the threaded PMMA insert 501 so that the outer surface of the bone is on the second offset line 512 and the described trialing process can be repeated. Once the proper PMMA insert 501 position is found, liquid PMMA cement can be applied to the PMMA insert, the bone and the implant. The liquid PMMA can cure forming a chemical bond with the PMMA insert and forming a mechanical bond between the bone and the implant. The cured liquid PMMA will also prevent the threaded implant from rotating which will effectively lock the PMMA insert in the offset position.

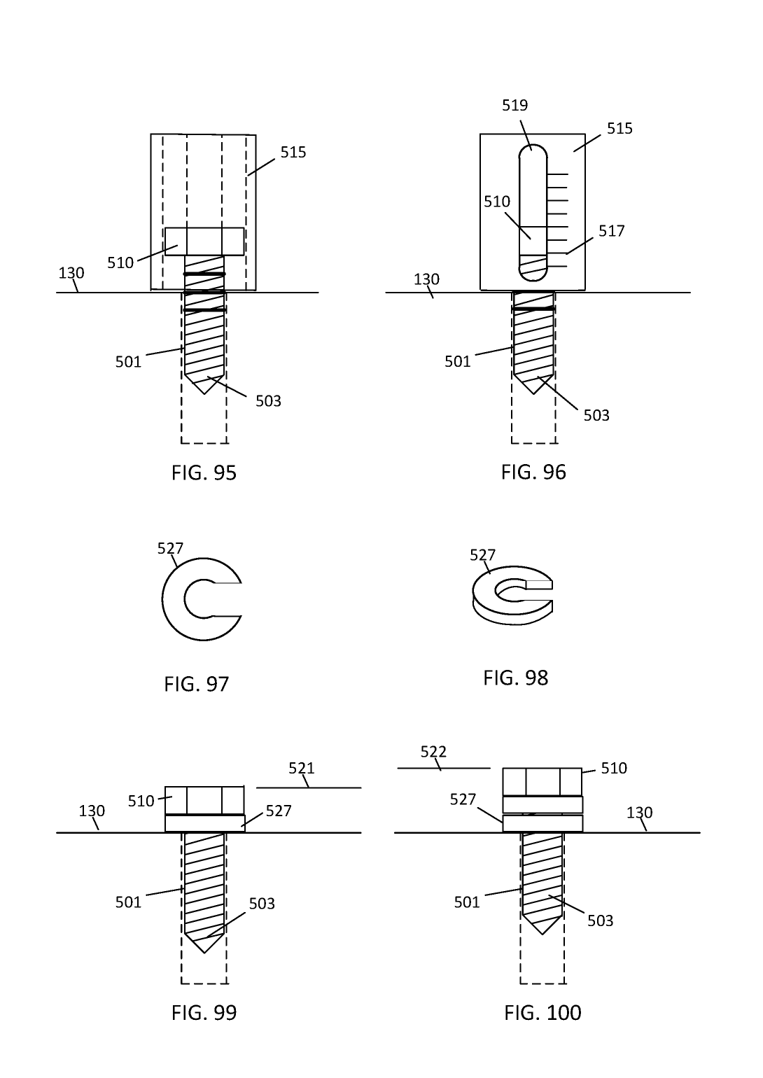

With reference to FIG. 95 a side view of a PMMA insert with the cap 510 in a rotational tool 515 is illustrated. In this example, the tool 515 may have a hexagonally shaped inner surface which fits over the hexagonal cap 510. The tool 515 can be rotated to rotate the PMMA insert 501 and drive it into the bone 130. With reference to FIG. 96, a side view of the rotational tool 515 is illustrated with a side window 519 and an offset visual scale 517. As the tool 515 rotates, the surgeon can monitor the position of the cap 510 and determine the offset of the cap 510 from the surface of the bone 130. Once the PMMA insert 501 is rotated to the desired offset, the tool 515 can be removed and a trial implant can be placed on the cap 510 of the PMMA insert 501. Trialing can be performed on the PMMA insert 501 and adjustments to the PMMA insert 501 can be made. Once the PMMA insert 501 is rotated to the desired offset, liquid PMMA cement can be applied to the PMMA insert and bone to prevent further rotation of the PMMA insert.

With reference to FIG. 97 a top view of a PMMA insert spacer 527 and in FIG. 98 a perspective view of a PMMA insert spacer 527 are illustrated. The spacers 527 can have a “C” shaped structure that can fit around the stem portion of the threaded PMMA insert and have uniform thicknesses. The surgeon can have a plurality of the spacers 527 available. With reference to FIG. 99, if the offset of the PMMA insert 527 needs to be increased, the PMMA insert 527 can be rotated to move the cap 510 away from the bone 130. A spacer 527 can then be placed around the stem 503 and between the bone 130 and the cap 510. The PMMA insert 501 can then be rotated to compress the spacer between the cap 510 and the bone 130. The offset 521 will be equal to the thickness of the spacer 527 and the cap 510 thickness. Trialing can be performed until the proper offset of the PMMA insert 501 is determined. With reference to FIG. 100, if additional offset is needed, an additional spacer(s) 527 can be used. The PMMA insert 501 can be rotated to move the cap 510 to fit another spacer 527 on the stem 503. The offset 522 will be equal to the thickness of two spacers 527 and the cap 510 thickness. Once the proper offset is found, the tool 515 can be removed, liquid PMMA can be applied to the bone 130, the spacers 527, the cured PMMA insert 510 and the implant. The liquid PMMA can cure to form a chemical bond with the spacers 527 and the insert 510. The cured PMMA components will also form a mechanical bond between the implant (not shown) and the bone 130.

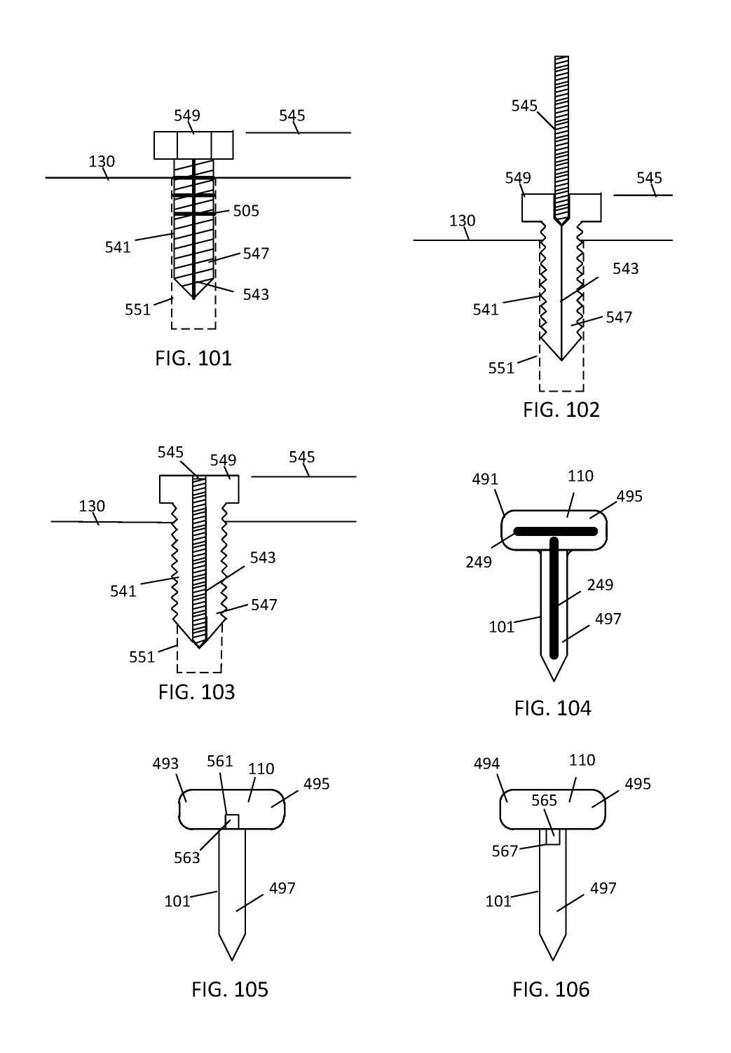

Another embodiment of a threaded PMMA insert 541 is illustrated in FIGS. 101-103. With reference to FIG. 101 a threaded PMMA insert 541 can be threaded into a hole 551 in a bone 130. In an embodiment, the cap 549 can have a hexagonal shape which can be rotated with a wrench or other tool. The stem 547 can include a slot 543 which can allow the stem to expand outward. The threaded PMMA insert 541 can have offset markings 505. The PMMA insert 541 can be rotated to a desired offset position and in this example, the first offset marking 505 can be aligned with the outer surface of the bone 130. With reference to FIG. 102, when the PMMA insert 541 is adjusted to the desired offset position, a expansion screw 545 can be threaded into the PMMA insert 541. The expansion screw 545 can have an internal hexagonal driver surface that can be rotated with a hex driver. With reference to FIG. 103, the expansion screw 545 can be threaded into the PMMA insert 541 and the stem 547 can be split at the slot 543 and pressed into the inner diameter surfaces of the hole 551.

In other embodiments, the tack inserts can have modular constructions. For example, in an embodiment the tack inserts can include cured PMMA stems and PMMA caps which can be assembled to create the tack inserts. With reference to FIGS. 104-106, examples of tack inserts that are modular designs fabricated with cured PMMA are illustrated. With reference to FIG. 104, a tack insert 491 can include a substrate 249 which can be an elongated rod made of a metal or non-PMMA polymer within a stem 101 and a PMMA 497 outer material. The cap 110 can be made of a PMMA material 495 covering a substrate 249. The proximal end of the substrate 249 in the stem 101 can be pressed into a hole in the cap 110 and the stem 101 can be bonded to the cap 110 with liquid PMMA cement. It can be more cost efficient to fabricate separate caps 110 and stems 101 and then assembly these components to create the tack inserts 491. These assembly modular PMMA inserts 491 can then be used as described above.

In yet another embodiment as shown in FIG. 105, a modular tack insert 493 can include the cap 110 component made of PMMA 495 and a stem 101 component made of PMMA 497. The cap 110 can have a hole 561 and the stem 101 can have a corresponding feature 563 that can be mechanically connected to the hole 561. A liquid PMMA cement can be used to chemically bond the stem 101 to the hole in the lower surface of the cap 110. When the tack insert 493 is pressed into the bone and liquid PMMA cement is applied, the cap 110 will be chemically bonded to the cured PMMA cement.

In FIG. 106, a tack insert 494 can also include the cap 110 component made of PMMA 495 and a stem 101 component made of PMMA material 497. The cap 110 can include a feature 565 on a lower surface which is chemically bonded to a hole 567 in the upper surface of the stem 101 with liquid PMMA cement. Again, when the tack insert 494 is pressed into the bone and liquid PMMA cement is applied, the cap 110 will be chemically bonded to liquid PMMA as it cures as described above.

As discussed, the inventive cured PMMA insert process can be used for various types of bone implants such as total knee arthroplasty. In an embodiment, the cured PMMA insert can be applied to bond the implant to the bone with the following surgical approach technique. An incision is made, the joint is exposed through one of several standard approaches through medial retinaculum and proximal extension. The surgeon can perform soft tissue releases and remove boney osteophytes. The surgeon then can prepare the patella, femur and tibia. The order of preparation of components can vary with the preference of the surgeon. In one common technique, the patella can be exposed and the thickness of the patella can be measured. The patella can then be cut and the knee can be sized for the implant. A drill guide can be applied to the patella and the patella can be prepared for the patella implant. A trial implant can be applied to the patella to check the fit of the final implant.

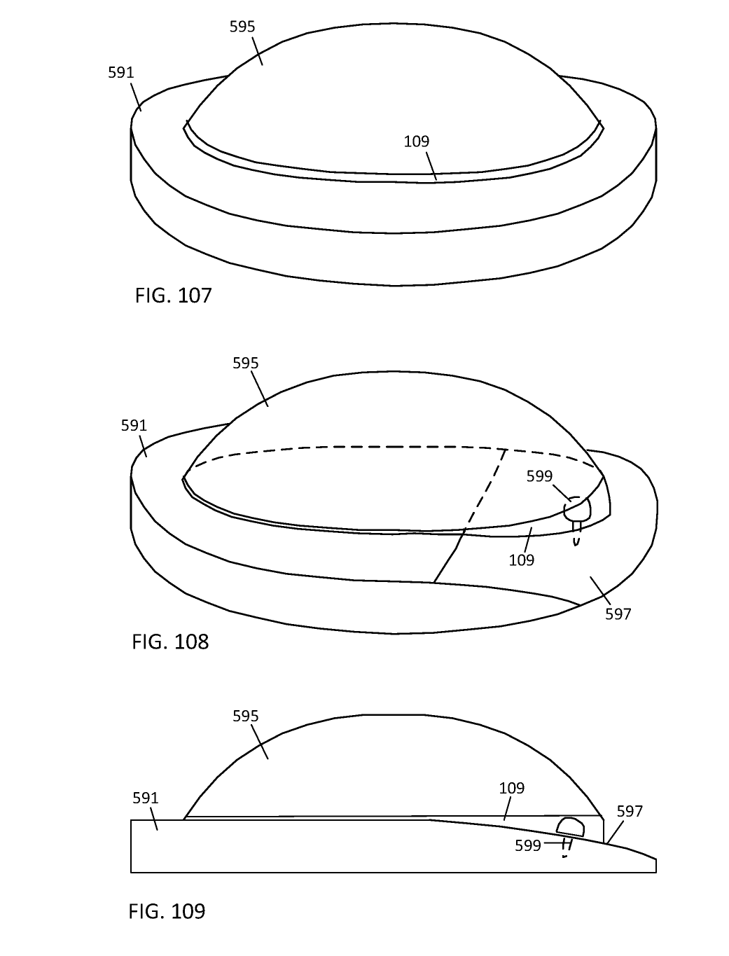

With reference to FIG. 107, a perspective view of a patella bone 591 is illustrated with a patella implant 595 secured to the patella bone 591 with liquid PMMA cement 109 is illustrated. As discussed, the patella bone 591 can be cut creating a planar resection surface. The patella implant 595 can have a convex outer surface and a planar lower surface that is secured to the resection surface of the patella bone 591. A perspective view of a patella bone 591, cured PMMA insert 599 and a patella implant 595 are illustrated with reference to FIG. 108. With reference to FIG. 109, a side view of the patella bone 591, cured PMMA insert 599 and a patella implant 595 are illustrated. In some patients, the patella bone 591 can have a damaged area 597 that needs to be built up and the resection of the patella bone 591 may not result in a planar surface upon which the patella implant 595 can be secured. In these situations, the described cured PMMA inserts can be used to properly secure the patella implant 595 to the patella bone 591. In the illustrated embodiment, a cured PMMA insert 599 can be placed in the damaged area 597 with the stem of the PMMA insert 599 pressed into the damaged area 597 of the patella bone 591 and a cap of the PMMA insert 599 in physical contact with the damaged area 597 and the patella implant 599. The surgeon can perform trialing on the patella insert 599 with a patella trial implant as described. When the proper PMMA insert 599 is found, liquid PMMA cement 109 can be applied to the patella implant 595, the patella bone 591 and the PMMA insert 599. The patella implant 595 can be placed on the patella bone 591 and the PMMA insert 599 which can provide physical support for the PMMA implant 599. The liquid PMMA cement 109 can cure to chemically bond to the PMMA insert 599 and create a mechanical bond between the patella implant 595 and the patella bone 591.

The trial implant is then removed and the femur is exposed, usually with two z type retractors. The distal femur is then drilled with an entry hole. An intramedullary rod and cutting guide can be applied to the distal femur. The guide can be adjusted for proper varus/valgus angle and to provide the proper amount of femoral bone resection. The cutting guide is then secured to the femur, usually with drill pins. The intramedullary rod and alignment jig are removed before cutting distal femur with oscillating bone saw. The femur can then be measured to determine the best bone implant size. The surgeon can then use the trial implant to determine the proper rotation of implant. The femur can be drilled to establish the joint rotation. A cutting guide can be used for making bone cuts with a bone saw. The cutting jig and bone fragments can then be removed from the surgical area.

The surgeon can then expose the tibia which can be done with medial, lateral and posterior retractors. The surgeon can then debride the meniscus and soft tissues. An extramedullary cutting guide can be applied to the anterior tibia. The guide can then be properly adjusted for: 1) amount of resection 2) posterior slope and 3) varus/valgus angle. The cutting guide can be secured to the tibia with pins. The tibia can be cut with an oscillating saw using the cutting guide. The cutting guide and bone fragments can then be removed. The surgeon can assess flexion and extension gaps with spacer blocks and determine if the joint is ready for a trial assessment. If the cuts appear correct, the surgeon can then apply and position trial components. Femoral component is applied to the femur followed by the tibial component, a tibial plastic spacer tray and the patellar component.

With the trial implants in place, the surgeon can perform a trial assessment.. Trial femoral, tibial and patellar implants can be tested to assess 1) tightness in extension and flexion and 2) medial and lateral soft tissue tension (looseness or tightness) throughout flexion and extension. The surgeon can assess the knee's range of motion and the stability of the knee throughout the range of motion. The tracking of the patella can also be assessed.

If range of motion demonstrates excess tightness or laxity, or if the knee is determined not to be adequately stable in any plane or any position, soft tissue balancing, recutting of bone or resizing of implants can be performed by the surgeon until proper range of motion and stability is achieved.

If the surgeon determines that augmentation of the bone is required or additional offset from the boney resected cuts, the surgeon can choose to use the insert(s) for correction. The inserts can be adjusted based upon the errors detected during trialing. With reference to Table 1 below, a listing of possible trial assessment imbalances are listed and the corresponding procedures for correcting the assessment defects.

TABLE 1

| Trial Assessment | |
|---|---|
| Imbalance | Possible Corrective Actions |
| Tight in extension | resecting more of the femur |
| | resecting the tibia with less slope |
| Tight in flexion only | Add tibial slope |
| | Reduce size of femoral component |
| Tight in flexion | Resect more tibia |
| and Extension | |
| Loose in | Recut tibia for more slope and add thickness of |
| Extension only | plastic insert |
| | Move Femoral Component distally with inserts |
| Loose in flexion | Increase size of femoral component (support post |
| only | surface with insert) |
| | Add thicker plastic insert, and resect more femur |
| Loose in Flexion | Use thicker plastic tibial insert. |
| and Extension | |
| Angular correction | Recut tibia |
| | Recut femur (cumbersome) |
| Excessive laxity | Tibia - More valgus cut and thicker poly with lateral |
| medially | release |
| | Make angular correction of femur with buildup |
| | using inserts |
| Excessive laxity | Tibia - medial release, varus recut, thicker poly |
| laterally | Femur - lateral distal buildup with inserts |

Choice of implants and proper preparation for placing final components is achieved when the knee demonstrates sufficient stability to varus and valgus stress throughout range of motion and the knee can move to full extension and full flexion, with good overall alignment of the limb.

The trial implant and any other trial devices can be removed. If inserts have been placed, the inserts can remain in place when the actual implant is bonded to the bone. The surgeon can irrigate the knee and remove soft tissue debris. At this stage, final preparation of the tibia is performed with drills and punches to set rotation and prepare for the stemmed component.

With the trialing complete, bone implants can be bonded to the tibia and/or femur. To bond the tibia implant, the tibia is exposed and the liquid PMMA cement is mixed. Liquid PMMA cement is applied and or pressurized to the tibial surface. The surgeon can impact the final tibial implant in place on the tibia, liquid PMMA and PMMA inserts. Once the final tibial implant is positioned, excess extruded PMMA cement is removed circumferentially about tibia and implant.

A similar process is used for bonding the femur implant. The bony surfaces of the femur are exposed. Pressurized liquid PMMA cement can be applied to the bony surfaces of the femur. The surgeon can then impact the final femoral implant in place on the femur, liquid PMMA and PMMA inserts. With the femoral implant properly positioned, the excess cement is circumferentially removed from the femur and implant. A tibial spacer can be placed and secured to the tibial component.

The patella can be exposed and liquid PMMA cement can be applied to the exposed patella. The final patellar implant component can be placed on the patella and the implant can be clamped to the patella. The excess cement can then be removed from the patella and patella implant.

After the liquid PMMA cement cures, the knee can be irrigated. The soft tissue around the knee can be closed and the skin can be closed. A dressing can be applied to the closed wound.

In the setting of revision total joint surgery, the surgeon can encounter deficiencies of any boney surface. The patella can be deficient and can provide only a shell of bone for fixation. Frequently, the surgeon is not able to cement a new patellar implant to the remaining bone. In the presence of deficiency, the surgeon can drill the patella and place one or more inserts that will support the patellar implant. The inserts are placed and secured to the patella. Liquid PMMA is applied to the undersurface of the patella and implant is applied to the cement, insert and patella. Excess cement is removed that is outside of the interface between the implant and bone. Implant is held in position until the PMMA cement had cured.

In revision total joint arthroplasty of joints other than the knee, there is frequently need to create offset or separation between bone and implant while cementing. In acetabular revision of the hip, in situations when a cup remains will ingrown to the acetabulum but no liner is available for the cup shell, surgeons can cement a polyethylene liner into the cup. It is commonly difficulty to accurately control the offset of the plastic liner from the metal shell. A large percentage of cups contain screw holes in the dome of the cup. For these cups, the surgeon can place inserts into the holes that create offset. The surgeon is then able to apply liquid cement and place a standard liner into the acetabular cup while maintaining offset to establish an adequate cement mantle. The surgeon also has the option of applying inserts or rods into a revision acetabulum and then cementing the acetabular component. The presence of inserts or rods can increase the strength of the bone cement interface and assist in accurate placement of the cup.

In the setting of revision total joint arthroplasty, it is common to use porous metal augments to fill large bone deficiencies. These can be used commonly in the acetabulum, the tibia or the femur. Standard technique calls for the surgeon to apply cement at the interface of the augment and the arthroplasty implant. The cement is placed to avoid mechanical wear of metal abrading metal surfaces. In this setting the surgeon can drill the porous metal augment and apply an insert or inserts to establish accurate separation of the two metal components. The insert can also provide improved mechanical loading between the two components by allowing pressure to be applied across the insert while the liquid cement cures. In shoulder arthroplasty, deficiencies of the glenoid bone stock can affect placement of a glenoid component. The described PMMA inserts can be used to offset the glenoid component relative to the glenoid bone.

### APPARATUS

In different embodiments, the cured PMMA inserts can have various configurations. For example, in an embodiment, an insert can provide an offset between a bone and an implant. The insert can comprise: an elongated stem made of a cured polymethyl methacrylate (PMMA) material defining an axis; and a cap made of the cured PMMA material in direct physical contact with the elongated stem wherein a distal surface of the cap is substantially perpendicular to the axis of the elongated stem. In some embodiments of the cured PMMA inserts can further comprise fenestrations formed in the elongated stem or the cap. The cured PMMA inserts can also have grooves formed in the elongated stem or the cap. The stem of the cured PMMA inserts can further comprise helical threads formed on an outer surface of the elongated stem and the cap includes drive surfaces which can be coupled to a rotational insertion tool such as a wrench, screw driver or any other driver tool.

Embodiments of the cured PMMA inserts can comprise just cured PMMA or have a composite construction that includes other materials. For example, in some embodiments the inserts can have a metal substrate within the elongated stem encapsulated within the PMMA material. Alternatively, the inserts a metal substrate within the cap encapsulated within the PMMA material. In some embodiments, the PMMA insert can include a polymer substrate. For example, the insert can have a polymer substrate within the elongated stem and or a cap encapsulated within the PMMA material.

In an embodiment, the PMMA insert can be a tack insert with a cap that has a first and a second surface which are on opposite sides of the cap. When the tack insert is pressed into the bone, a first surface of the cap is adapted for direct physical contact with a resection surface of a bone and a second surface of the cap opposite the first surface is adapted for contact with a bone implant as discussed in the embodiments of the tack insert described above. In some embodiments the upper and lower surfaces of the cap can be planar and parallel. However, in other embodiments, the upper and lower surfaces of the cap, can be non-parallel and form an acute angle.

In some embodiments, the cured PMMA insert can be a shim which provides an offset between a bone and an implant. The shim insert can comprise an elongated stem made of a cured PMMA material. The stem can define an axis. A head made of the cured PMMA material is in direct physical contact with the elongated stem wherein a distal surface of the head can be substantially perpendicular to the axis of the elongated stem. In some embodiments, the PMMA shim insert can have a composite construction that includes a metal or a polymer substrate in the head and/or stem encapsulated within the PMMA material. In some embodiments, the cured PMMA shim insert can include fenestrations formed in the elongated stem or the head. In some embodiments, the insert can include grooves formed in the elongated stem or the head. The head can have upper and lower surfaces which can be planar and parallel. Alternatively, the upper and lower surfaces of the head can be non-parallel where the upper and lower surfaces form an acute angle.

The present disclosure, in various embodiments, includes components, and apparatus substantially as depicted and described herein, including various embodiments, sub-combinations, and subsets thereof. Those of skill in the art will understand how to make and use the present disclosure after understanding the present disclosure. The present disclosure, in various embodiments, includes providing devices and processes in the absence of items not depicted and/or described herein or in various embodiments hereof, including in the absence of such items as may have been used in previous devices or processes, e.g., for improving performance, achieving ease and/or reducing cost of implementation. Rather, as the flowing claims reflect, inventive aspects lie in less than all features of any single foregoing disclosed embodiment.

1. A method for securing an implant to a bone of a patient comprising:
providing a cured polymethyl methacrylate (PMMA) insert having a stem;
inserting the stem of the PMMA insert into a resection surface of the bone wherein a portion of the PMMA insert extends out of the resection surface of the bone;
placing the implant against the portion of the PMMA insert extending from the resection surface of the bone to maintain a fixed length offset of the implant relative to the resection surface of the bone;
applying a liquid PMMA to the PMMA insert, the bone and the implant; and
curing the liquid PMMA to chemically bond to the PMMA insert and mechanically secure the implant to the bone.

2. (canceled)

3. (canceled)

4. (canceled)

5. The method of claim 1 further comprising:
filling a volume between the bone and the implant with liquid PMMA.

6. (canceled)

7. (canceled)

8. The method of claim 1 further comprising:
fabricating the PMMA insert by encapsulating a metal substrate or a polymer substrate within cured PMMA.

9. (canceled)

10. (canceled)

11. (canceled)

12. (canceled)

13. (canceled)

14. The method of claim 9 wherein the stem has a tapered cross section with a smaller cross section at a tip of the stem than the base of the stem at the junction of the cap and wherein the tapered cross section expands outward against the resection surface of the bone as the stem is inserted into the resection surface of the bone.

15. The method of claim 1 wherein the PMMA insert has a head in direct physical contact with the stem, a first surface of the head contacts the resection surface of the bone and a second surface of the head opposite the first surface contacts the implant during the placing step.

16. (canceled)

17. The method of claim 15 further comprising:
providing a second cured PMMA insert having a second stem, wherein the second cured PMMA insert has a second head in direct physical contact with the second stem;
inserting the second stem of the second PMMA insert into a second resection surface of the bone wherein the second head extends out of the second resection surface, wherein a first surface of the second head contacts the second resection surface of the bone;
placing the implant against the portion of the second PMMA insert extending from the second resection surface of the bone, wherein a second surface of the second head opposite the first surface contacts the implant;
applying a liquid PMMA to the second PMMA insert with the bone and the implant; and
curing the liquid PMMA to bond the second PMMA insert to the bone and the implant.

18. (canceled)

19. The method of claim 1 further comprising:
providing a second cured PMMA insert having a stem;
inserting the stem of the second PMMA insert into a second resection surface of a bone wherein a portion of the second PMMA insert extends out of the second resection surface of the second bone surface;
placing the implant against the portion of the second PMMA insert extending from the second resection surface of the bone to maintain a second fixed length offset of the implant relative to the second resection surface of the bone;
applying a liquid PMMA to the second PMMA insert, the bone and the implant; and
curing the liquid PMMA to bond the second PMMA insert to the bone and the implant.

20. The method of claim 19 wherein the bone is a femur, the resection surface is on a medial condyle of the femur (MFC) and the second resection surface is on a lateral femoral condyle of the femur (LFC).

21. The method of claim 19 wherein the bone is a femur and the implant is a femoral component of a replacement knee, the resection surface is on a medial condyle of the femur (MFC) and the second resection surface is on a lateral femoral condyle of the femur (LFC), the fixed length offset of the implant relative to the resection surface on the MFC and the second fixed length offset of the implant relative to the second resection surface on the LFC cause the implant to be offset longitudinally relative to the femur.

22. The method of claim 19 wherein the bone is a femur, the resection surface is on a medial condyle of the femur (MFC) and the second resection surface is on a lateral femoral condyle of the femur (LFC), the fixed length offset of the implant relative to the resection surface on the MFC and the second fixed length offset of the implant relative to the second resection surface on the LFC are equal, causing the implant to be longitudinally offset by a fixed distance relative to the femur.

23. The method of claim 19 wherein the bone is a femur, the resection surface is on a medial condyle of the femur (MFC) and the second resection surface is on a lateral femoral condyle of the femur (LFC), the fixed length offset of the implant relative to the resection surface on the MFC and the second fixed length offset of the implant relative to the second resection surface on the LFC are not equal, causing the implant to be offset angularly relative to a center axis of the femur.

24. (canceled) The method of claim 19 wherein the fixed length offset of the implant relative to the resection surface on the MFC and the second fixed length offset of the implant relative to the second resection surface on the LFC offset the implant are equal, causing the implant to be offset in a distal direction relative to a joint line of a limb.

25. The method of claim 19 wherein the fixed length offset of the implant relative to the resection surface on the MFC and the second fixed length offset of the implant relative to the second resection surface on the LFC offset the implant are not equal, causing the implant to be offset at a predefined angular orientation relative to the bone.

26. A method for securing an implant to a bone of a patient comprising:
providing a first cured polymethyl methacrylate (PMMA) insert having a first stem;
inserting the first stem of the first PMMA insert into a resection surface of the bone wherein a first portion of the first PMMA insert extends out of the resection surface of the bone;
determining that the first portion of the first PMMA insert that extends out of the resection surface of the bone is not a proper length to maintain a predetermined proper fixed length offset of the implant relative to the resection surface of the bone;
removing the first PMMA implant from the resection surface of the bone;
providing a second cured PMMA insert having a second stem, wherein an offset of a of the second PMMA insert is different than an offset of the first portion of the first PMMA insert;
inserting the second stem of the PMMA insert into the resection surface of the bone;
determining that the second portion of the second PMMA insert that extends out of the resection surface of the bone is the proper length to maintain the proper fixed length offset of the implant relative to the resection surface of the bone;
placing the implant against the second portion of the second PMMA insert extending from the resection surface of the bone;
applying a liquid PMMA to the second PMMA insert, the bone and the implant; and
curing the liquid PMMA to chemically bond to the second PMMA insert and mechanically secure the implant to the bone.

27. (canceled)

28. (canceled)

29. (canceled)

30. The method of claim 26 further comprising:
filling a volume between the bone and the implant with liquid PMMA.

31. (canceled)

32. The method of claim 26 wherein the first PMMA insert and/or the second PMMA insert is a tack having a cap in direct physical contact with the stem, a first surface of the cap is placed in direct physical contact with the resection surface of the bone during the inserting steps and a second surface of the cap opposite the first surface contacts the implant during the placing step.

33. The method of claim 26 further comprising:
fabricating the first PMMA insert and the second PMMA insert by encapsulating a metal substrate or a polymer substrate within cured PMMA.

34. The method of claim 26 wherein the first PMMA insert is a shim having a first head in direct physical contact with the first stem, a first surface of the first head is in direct physical contact with the resection surface of the bone during the inserting steps and a second surface of the first head opposite the first surface contacts the implant during the placing step.

35. The method of claim 30 wherein the first surface and the second surface of the first head of the first shim are non-parallel surfaces that define an acute angle.

36. The method of claim 30 wherein the second PMMA insert is a second shim having a second head in direct physical contact with the second stem, a first surface of the second head is in direct physical contact with the resection surface of the bone during the inserting steps and a second surface of the head opposite the first surface contacts the implant during the placing step.

37. (canceled)

38. A method for securing an implant to an exposed bony surface of a bone of a patient comprising:
providing a first cured polymethyl methacrylate (PMMA) insert having a first stem and a second PMMA insert having a second stem;
inserting the first stem of the first PMMA insert into the exposed bony surface of the bone;
inserting the second stem of the second PMMA insert into the exposed bony surface of the bone;
applying a liquid PMMA to the first PMMA insert, the second PMMA insert and the bone;
placing the implant against the liquid PMMA that is also in contact with the first PMMA insert and the second PMMA insert; and
curing the liquid PMMA to chemically bond to the first PMMA insert and the second PMMA and secure the implant to the bone.

39. (canceled)

40. The method of claim 38 further comprising:
pressurizing the liquid PMMA; and
injecting the liquid PMMA into a gap between the exposed bony surface and the implant.

41. The method of claim 38 wherein orientations of the first stem and the second stem are not parallel within the exposed bony surface.

42. (canceled)

43. (canceled)

44. (canceled)

45. (canceled)

46. The method of claim 38 wherein the bone is a femur and the exposed bony surface is in a femoral metaphysis.

47. (canceled)

48. The method of claim 38 wherein the inserting steps each include drilling a hole in the exposed boney surface of the bone and inserting the first stem of the first PMMA insert or the second stem of the second PMMA insert into the hole.

49. The method of claim 38 further comprising:
filling a volume between the exposed bony surface of the bone and the implant with liquid PMMA.

50. (canceled)

51. The method of claim 38 wherein the first PMMA insert is a first tack having a first cap coupled to the first stem and the second PMMA insert is a second tack having a second cap coupled to the second stem, wherein first surfaces of the first cap and the second cap contact the exposed bony surface of the bone during the inserting steps and second surface of the first cap and the second cap opposite the first surfaces contact the implant during the placing step.

52. (canceled)

53. (canceled)

54. (canceled)

55. The method of claim 51 wherein the first stem and the second stem each have an anchor mechanism which resists the removal of the first stem and the second stem from the exposed boney surface of the bone after the first stem and the second stem have been inserted into the exposed boney surface.

56. The method of claim 51 wherein the first stem and the second stem each have a tapered cross section along the length of the first stem and the second stem and wherein the tapered cross section is pressed outward against the exposed bony surface of the bone as the first stem and the second stem during the inserting steps.

57. The method of claim 38 wherein the bone is a tibia and the exposed bony surface is in a tibial metaphysis.

58. The method of claim 38 wherein the bone is a pelvis and the implant is an acetabular liner or a shell.

59. (canceled)

60. (canceled)

61. (canceled)

62. (canceled)
